(19) United States
(12) Patent Application Publication
(10) Pub. No.: US 2025/0049710 A1
(43) Pub. Date: Feb. 13, 2025

(54) AQUEOUS FORMULATIONS OF A CARDIAC P2X RECEPTOR AGONIST AND THEIR USE IN TREATING MEDICAL DISORDERS

(71) Applicant: Comovus Pharmaceuticals, Inc., Farmington, CT (US)

(72) Inventors: Bruce T. Liang, Avon, CT (US); Mingliu Lei, Hangzhou (CN); Hongjuan Xue, Dingxi (CN); Yinan Han, Zhoukoucn (CN); Wenjie Song, Huanggang (CN); Menghua Li, Xuchang Henan (CN)

(21) Appl. No.: 18/778,671

(22) Filed: Jul. 19, 2024

(30) Foreign Application Priority Data
Jul. 21, 2023 (WO) PCT/CN2023/108504

(51) Int. Cl. A61K 9/08; A61K 31/675; A61K 47/02; A61K 47/12; A61K 47/18; A61K 47/26

(52) U.S. Cl. CPC A61K 9/08; A61K 31/675; A61K 47/02; A61K 47/12; A61K 47/183; A61K 47/26

(57) ABSTRACT

The invention provides aqueous formulations containing a compound that stimulates cardiac P2X receptor activity, and methods for their use in stimulating cardiac P2X receptor activity and treating medical disorders, such as heart failure.

### CROSS REFERENCE TO RELATED APPLICATIONS

This application claims the benefit of and priority to patent application serial number PCT/CN2023/108594, filed Jul. 21, 2023, the contents of which are hereby incorporated by reference.

### FIELD OF THE INVENTION

The invention provides aqueous formulations containing a compound that stimulates cardiac P2X receptor activity, and methods for their use in stimulating cardiac P2X receptor activity and treating medical disorders, such as heart failure.

### BACKGROUND

Heart failure affects a substantial number of patients worldwide and is characterized by impairment of the heart's ability to pump blood. Exemplary symptoms of heart failure include, for example, shortness of breath, excessive fatigue, and leg swelling. Coronary artery disease, heart attack, high blood pressure, atrial fibrillation, valvular heart disease, excessive alcohol consumption, infection, and cardiomyopathy are factors that increase a patient's risk for experiencing heart failure.

Stimulation of cardiac P2X receptors has been reported to provide benefits to subjects suffering from heart failure. See, for example, Zhou et al. in J. Pharmacol. Exp. Ther. (2010) vol. 333 (3), pages 920-928. P2X receptors are expressed in cardiac tissue, and cardiac P2X receptor stimulation causes an increase of nitric oxide and cyclic guanosine monophosphate (cGMP) in cardiomyocytes. Additional compounds and therapeutic methods using a cardiac P2X receptor agonist are described in U.S. Pat. Nos. 9,303,053 and 9,526,739.

New formulations for treating heart failure that are stable and ready-to-use for combining with a diluent for intravenous administration are needed and would benefit patients. The present invention addresses this need and provides other related advantages.

### SUMMARY

The invention provides aqueous formulations containing a compound that stimulates cardiac P2X receptor activity, and methods for their use in stimulating cardiac P2X receptor activity and treating medical disorders, such as heart failure. The aqueous formulations demonstrate superior stability to storage, and the aqueous formulations are ready-to-use for mixing with a diluent for intravenous administration to provide an aqueous injectable formulation. The aqueous injectable formulation can be administered to a patient by intravenous injection. These and other aspects of the invention aqueous formulations and aqueous injectable formulations and medical uses are described herein below.

Accordingly, one aspect of the invention provides an aqueous formulation, comprising:
(a) from 0.25% (w/v) to 0.75% (w/v) of a compound of Formula I, wherein Formula I is represented by:

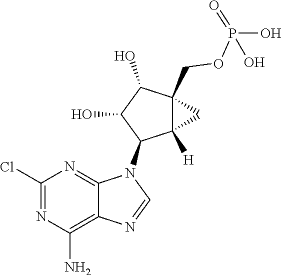 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof;
(b) from 0.01% (w/v) to 0.03% (w/v) of a chelating agent selected from ethylenediamine tetraacetic acid, a pharmaceutically acceptable salt of ethylenediamine tetraacetic acid, or a mixture thereof;
(c) from 10 mM to 30 mM of a buffer comprising citric acid and a pharmaceutically acceptable salt of citric acid;
(d) from 3.5% (w/v) to 11% (w/v) of a tonicity modifier; and
(e) at least 90% (w/v) water; and
having a pH in the range of 7.5 to 8.5.

Another aspect of the invention provides an aqueous formulation, comprising:
(a) about 0.5% (w/v) of a compound of Formula I, wherein Formula I is represented by:

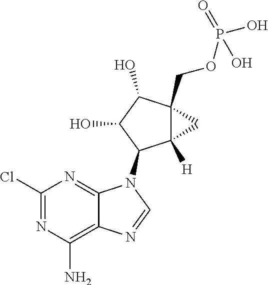 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof;
(b) about 0.02% (w/v) of a chelating agent selected from ethylenediamine tetraacetic acid, a pharmaceutically acceptable salt of ethylenediamine tetraacetic acid, or a mixture thereof;
(c) about 20 mM of a buffer comprising citric acid and a pharmaceutically acceptable salt of citric acid;
(d) about 6.9% (w/v) of a tonicity modifier; and
(e) at least 90% (w/v) water; and
having a pH of about 8.

Further description of additional aqueous formulations is described in the detailed description. For example, another aspect of the invention provides an aqueous formulation, comprising:
(a) from 0.25% (w/v) to 0.75% (w/v) of a compound of Formula I, wherein Formula I is represented by:

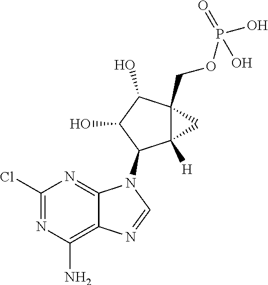 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof;
(b) from 0.01% (w/v) to 0.06% (w/v) of a chelating agent;
(c) a buffer; and
(d) at least 85% (w/v) water; and
having a pH in the range of 7.0 to 8.5.

Another aspect of the invention provides an aqueous injectable formulation comprising an aqueous formulation described herein and a diluent for intravenous administration. Exemplary diluents for intravenous administration include saline solution and Lactated Ringer's solution. Another aspect of the invention provides an aqueous injectable formulation produced by admixing a formulation described herein and a diluent for intravenous administration. Another aspect of the invention provides a method of preparing an aqueous injectable formulation, comprising admixing a formulation described herein and a diluent for intravenous administration.

Another aspect of the invention provides a method of treating a cardiac disorder, wherein the method comprises administering to a subject in need thereof a therapeutically effective amount of an aqueous injectable formulation described herein to treat the cardiac disorder.

Another aspect of the invention provides a method for improving cardiac contractile performance in a subject, wherein the method comprises administering to a subject in need thereof an effective amount of an aqueous injectable formulation described herein to improve cardiac contractile performance.

Another aspect of the invention provides a method for improving cardiac function in a subject, wherein the method comprises administering to a subject in need thereof an effective amount of an aqueous injectable formulation described herein to improve cardiac function.

Another aspect of the invention provides a method of stimulating the activity of a cardiac P2X receptor in a subject, comprising administering to the subject in need thereof an effective amount of an aqueous injectable formulation described herein to stimulate the activity of said cardiac P2X receptor.

### DETAILED DESCRIPTION

The invention provides aqueous formulations containing a compound that stimulates cardiac P2X receptor activity, and methods for their use in stimulating cardiac P2X receptor activity and treating medical disorders, such as heart failure. The aqueous formulations demonstrate superior stability to storage, and the aqueous formulations are ready-to-use for mixing with a diluent for intravenous administration to provide an aqueous injectable formulation. The aqueous injectable formulation can be administered to a patient by intravenous injection. These and other aspects of the invention aqueous formulations and aqueous injectable formulations and medical uses are described herein below. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In certain embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In certain embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl) alkyl or (cycloalkyl) alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In certain embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In certain embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

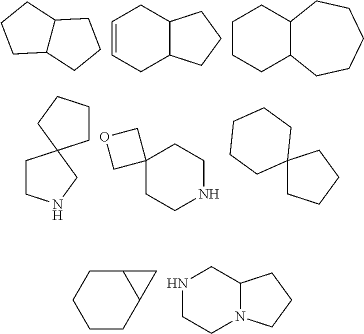

Exemplary bridged bicyclics include:

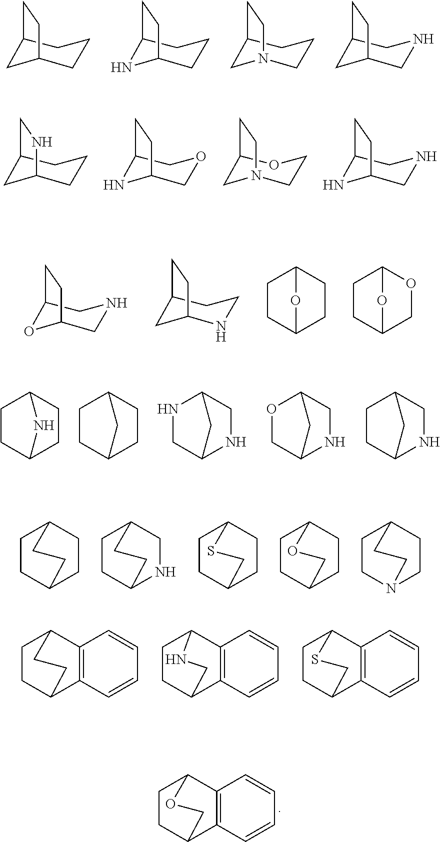

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H pyrrolyl), NH (as in pyrrolidinyl), or NR (as in N substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by an oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•}.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR^{•}₂, =NNHC(O)R^{•}, =NNHC(O)OR^{•}, ═NNHS(O)₂R^{•}, =NR^{•}, =NOR^{•}, —O(C(R^{•}₂))₂₋₃O—, or —S(C(R^{•}₂))₂₋₃S—, or a divalent substituent bound to vicinal substitutable carbons of an “optionally substituted” group is —O(CR^{•}₂)₂₋₃O—, wherein each independent occurrence of R^{•} is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R^{•} is C₁₋₆ aliphatic, R^{•} is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, RT is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley V C H; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

Unless specified otherwise, the term “about” refers to within ±10% of the stated value. The invention encompasses embodiments where the value is within ±9%, ±8%, ±7%, ±6%, ±5%, ±4%, ±3%, ±2%, or ±1% of the stated value.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The terms “alkenyl” and “alkynyl” are art-recognized and refer to unsaturated aliphatic groups analogous in length and possible substitution to the alkyls described above, but that contain at least one double or triple bond respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like. The term “hydroxyalkoxyl” refers to an alkoxyl group that is substituted with at least one hydroxyl. Exemplary hydroxyalkoxyl groups include —OCH₂CH₂OH, —OCH₂C(H)(OH)CH₂CH₂OH, and the like. The term “alkoxylene” refers to a bivalent alkoxyl group.

The term “oxo” is art-recognized and refers to a “=O” substituent. For example, a cyclopentane substituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and, most preferably, includes humans.

As used herein, the term “compound” refers to a quantity of molecules that is sufficient to be weighed, tested for its structural identity, and to have a demonstrable use (e.g., a quantity that can be shown to be active in an assay, an in vitro test, or in vivo test, or a quantity that can be administered to a patient and provide a therapeutic benefit).

As used herein, the term “effective amount” refers to the amount of a compound sufficient to effect beneficial or desired results (e.g., a therapeutic, ameliorative, inhibitory, or preventative result). An effective amount can be administered in one or more administrations, applications, or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Aqueous Formulations

The invention provides aqueous formulations containing a cardiac P2X receptor agonist. The aqueous formulations may be combined with a diluent for intravenous administration, and the resulting aqueous injectable formulation may be used in the therapeutic methods described herein. Exemplary aqueous formulations are described in the following sections.

### Part A—First Aqueous Formulation

One aspect of the invention provides an aqueous formulation, comprising:
(a) from 0.25% (w/v) to 0.75% (w/v) of a compound of Formula I, wherein Formula I is represented by:

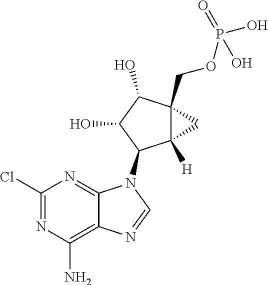 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) from 0.01% (w/v) to 0.03% (w/v) of a chelating agent selected from ethylenediamine tetraacetic acid, a pharmaceutically acceptable salt of ethylenediamine tetraacetic acid, or a mixture thereof;
(c) from 10 mM to 30 mM of a buffer comprising citric acid and a pharmaceutically acceptable salt of citric acid;
(d) from 3.5% (w/v) to 11% (w/v) of a tonicity modifier; and
(e) at least 90% (w/v) water; and having a pH in the range of 7.5 to 8.5.

The First Aqueous Formulation may be further described according to additional features, such as the amounts and identities of constituents in the formulation. A more thorough description of such features is provided below. The invention embraces all permutations and combinations of these features.

In certain embodiments, the compound of Formula I is present in an amount of from 0.4% (w/v) to 0.6% (w/v) in the formulation. In certain embodiments, the compound of Formula I is present in an amount of about 0.5% (w/v) in the formulation. In certain embodiments, the compound of Formula I is present in an amount of 0.5% (w/v) in the formulation.

In yet other embodiments, the formulation comprises from 0.25% to 0.75% (w/v); 0.25% to 0.70% (w/v); 0.25% to 0.65% (w/v); 0.25% to 0.60% (w/v); 0.25% to 0.55% (w/v); 0.25% to 0.50% (w/v); 0.25% to 0.45% (w/v); 0.25% to 0.40% (w/v); 0.25% to 0.35% (w/v); 0.25% to 0.30% (w/v); 0.30% to 0.75% (w/v); 0.30% to 0.70% (w/v); 0.30% to 0.65% (w/v); 0.30% to 0.60% (w/v); 0.30% to 0.55% (w/v); 0.30% to 0.50% (w/v); 0.30% to 0.45% (w/v); 0.30% to 0.40% (w/v); 0.30% to 0.35% (w/v); 0.35% to 0.75% (w/v); 0.35% to 0.70% (w/v); 0.35% to 0.65% (w/v); 0.35% to 0.60% (w/v); 0.35% to 0.55% (w/v); 0.35% to 0.50% (w/v); 0.35% to 0.45% (w/v); 0.35% to 0.40% (w/v); 0.40% to 0.75% (w/v); 0.40% to 0.70% (w/v); 0.40% to 0.65% (w/v); 0.40% to 0.60% (w/v); 0.40% to 0.55% (w/v); 0.40% to 0.50% (w/v); 0.40% to 0.45% (w/v); 0.45% to 0.75% (w/v); 0.45% to 0.70% (w/v); 0.45% to 0.65% (w/v); 0.45% to 0.60% (w/v); 0.45% to 0.55% (w/v); 0.45% to 0.50% (w/v); 0.50% to 0.75% (w/v); 0.50% to 0.70% (w/v); 0.50% to 0.65% (w/v); 0.50% to 0.60% (w/v); 0.50% to 0.55% (w/v); 0.55% to 0.75% (w/v); 0.55% to 0.70% (w/v); 0.55% to 0.65% (w/v); 0.55% to 0.60% (w/v); 0.60% to 0.75% (w/v); 0.60% to 0.70% (w/v); 0.60% to 0.65% (w/v); 0.65% to 0.75% (w/v); 0.65% to 0.70% (w/v); or 0.70% to 0.75% (w/v) of a compound of Formula I.

In certain embodiments, the formulation comprises about 0.25%, 0.30%, 0.35%, 0.40%, 0.45%, 0.50%, 0.55%, 0.60%, 0.65%, 0.70%, or 0.75% (w/v) of a compound of Formula I. In certain embodiments of the formulation, a compound of Formula I is present in an amount of 0.25%, 0.30%, 0.35%, 0.40%, 0.45%, 0.50%, 0.55%, 0.60%, 0.65%, 0.70%, or 0.75% (w/v).

In certain embodiments of the formulations, the compound of Formula I is a pharmaceutically acceptable salt of

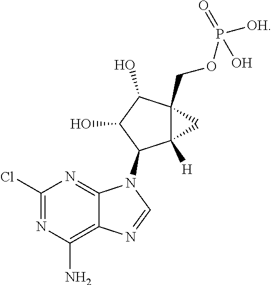 [SMILES: Nc1nc(Cl)nc2c1ncn2[C@H]1[C@H](O)[C@H](O)[C@]2(COP(=O)(O)O)C[C@H]12]

For instance, the compound of Formula I may be an alkali metal or alkaline earth metal pharmaceutically acceptable salt. In certain embodiments, the compound of Formula I is a sodium salt.

The formulation may be characterized according to the chelating agent. For instance, the chelating agent may be ethylenediamine tetraacetic acid (EDTA), a pharmaceutically acceptable salt of ethylenediamine tetraacetic acid, or a mixture thereof. In certain embodiments, the chelating agent is EDTA. In certain embodiments, the chelating agent is a pharmaceutically acceptable salt of EDTA. In certain embodiments, the chelating agent is a mixture of EDTA and a pharmaceutically acceptable salt of EDTA.

In certain embodiments, the formulation comprises from 0.01% to 0.03% (w/v) of a chelating agent. For instance, in certain embodiments, the formulation comprises 0.015% (w/v) to 0.025% (w/v) of a chelating agent selected from ethylenediamine tetraacetic acid, a pharmaceutically acceptable salt of ethylenediamine tetraacetic acid, or a mixture thereof. In certain embodiments, the formulation comprises about 0.02% (w/v) of a chelating agent selected from ethylenediamine tetraacetic acid, a pharmaceutically acceptable salt of ethylenediamine tetraacetic acid, or a mixture thereof. In certain embodiments, the formulation comprises 0.02% (w/v) of a chelating agent selected from ethylenediamine tetraacetic acid, a pharmaceutically acceptable salt of ethylenediamine tetraacetic acid, or a mixture thereof.

In certain embodiments, the formulation comprises from 0.01% to 0.03% (w/v); 0.01% to 0.025% (w/v); 0.01% to 0.02% (w/v); 0.01% to 0.015% (w/v); 0.015% to 0.03% (w/v); 0.015% to 0.025% (w/v); 0.015% to 0.02% (w/v); 0.02% to 0.03% (w/v); 0.02% to 0.025% (w/v); or 0.025% to 0.03% (w/v) of a chelating agent. In certain embodiments, the formulation comprises about 0.01%, 0.015%, 0.02%, 0.025%; or 0.03% (w/v) of a chelating agent. In certain embodiments, the formulation comprises 0.01%, 0.015%, 0.02%, 0.025%; or 0.03% (w/v) of a chelating agent.

The formulation may be characterized according to the buffer. In certain embodiments, the formulation comprises from 15 mM to 25 mM of a buffer comprising citric acid and a pharmaceutically acceptable salt of citric acid. In certain embodiments, the formulation comprises from 18 mM to 22 mM of a buffer comprising citric acid and a pharmaceutically acceptable salt of citric acid. In certain embodiments, the formulation comprises about 20 mM of a buffer comprising citric acid and a pharmaceutically acceptable salt of citric acid. In certain embodiments, the formulation comprises 20 mM of a buffer comprising citric acid and a pharmaceutically acceptable salt of citric acid. In certain embodiments, the formulation comprises 10, 11, 12, 13, 14, 15, 16, 17, 18, 19, 20, 21, 22, 23, 24, 25, 26, 27, 28, 29 or 30 mM of a buffer comprising citric acid and a pharmaceutically acceptable salt of citric acid.

In certain embodiments, the formulation comprises from 0.25% (w/v) to 0.75% (w/v) of a buffer comprising citric acid and a pharmaceutically acceptable salt of citric acid. For instance, in certain embodiments, the formulation comprises from 0.25% to 0.75% (w/v); 0.25% to 0.70% (w/v); 0.25% to 0.65% (w/v); 0.25% to 0.60% (w/v); 0.25% to 0.55% (w/v); 0.25% to 0.50% (w/v); 0.25% to 0.45% (w/v); 0.25% to 0.40% (w/v); 0.25% to 0.35% (w/v); 0.25% to 0.30% (w/v); 0.30% to 0.75% (w/v); 0.30% to 0.70% (w/v); 0.30% to 0.65% (w/v); 0.30% to 0.60% (w/v); 0.30% to 0.55% (w/v); 0.30% to 0.50% (w/v); 0.30% to 0.45% (w/v); 0.30% to 0.40% (w/v); 0.30% to 0.35% (w/v); 0.35% to 0.75% (w/v); 0.35% to 0.70% (w/v); 0.35% to 0.65% (w/v); 0.35% to 0.60% (w/v); 0.35% to 0.55% (w/v); 0.35% to 0.50% (w/v); 0.35% to 0.45% (w/v); 0.35% to 0.40% (w/v); 0.40% to 0.75% (w/v); 0.40% to 0.70% (w/v); 0.40% to 0.65% (w/v); 0.40% to 0.60% (w/v); 0.40% to 0.55% (w/v); 0.40% to 0.50% (w/v); 0.40% to 0.45% (w/v); 0.45% to 0.75% (w/v); 0.45% to 0.70% (w/v); 0.45% to 0.65% (w/v); 0.45% to 0.60% (w/v); 0.45% to 0.55% (w/v); 0.45% to 0.50% (w/v); 0.50% to 0.75% (w/v); 0.50% to 0.70% (w/v); 0.50% to 0.65% (w/v); 0.50% to 0.60% (w/v); 0.50% to 0.55% (w/v); 0.55% to 0.75% (w/v); 0.55% to 0.70% (w/v); 0.55% to 0.65% (w/v); 0.55% to 0.60% (w/v); 0.60% to 0.75% (w/v); 0.60% to 0.70% (w/v); 0.60% to 0.65% (w/v); 0.65% to 0.75% (w/v); 0.65% to 0.70% (w/v); or 0.70% to 0.75% (w/v) of a buffer comprising citric acid and a pharmaceutically acceptable salt of citric acid.

In certain embodiments, the formulation comprises about 0.25%, 0.30%, 0.35%, 0.40%, 0.45%, 0.50%, 0.55%, 0.60%, 0.65%, 0.70%, or 0.75% (w/v) of a buffer comprising citric acid and a pharmaceutically acceptable salt of citric acid. In certain embodiments of the formulation, a buffer comprising citric acid and a pharmaceutically acceptable salt of citric acid is present in an amount of 0.25%, 0.30%, 0.35%, 0.40%, 0.45%, 0.50%, 0.55%, 0.60%, 0.65%, 0.70%, or 0.75% (w/v). In certain embodiments, the formulation comprises 0.56% or about 0.56% (w/v) of a buffer comprising citric acid and a pharmaceutically acceptable salt of citric acid.

The formulations may be further characterized according to the tonicity modifier. For instance, in certain embodiments, the formulation comprises from 3.5% to 11% (w/v) of a tonicity modifier. In certain embodiments, the formulation comprises from 3.5% (w/v) to 9% (w/v) of a tonicity modifier. In certain embodiments, the formulation comprises from 4% (w/v) to 9% (w/v) of a tonicity modifier. In certain embodiments, the formulation comprises from 5% (w/v) to 9% (w/v) of a tonicity modifier. In certain embodiments, the formulation comprises from 6% (w/v) to 8% (w/v) of a tonicity modifier. In certain embodiments, the formulation comprises about 6.9% (w/v) of a tonicity modifier. In certain embodiments, the formulation comprises 6.9% (w/v) of a tonicity modifier.

In certain embodiments, the formulation comprises from 3.5% to 11%; 3.5% to 10.5%; 3.5% to 10%; 3.5% to 9.5%; 3.5% to 9%; 3.5% to 8.5%; 3.5% to 8%; 3.5% to 7.5%; 3.5% to 7%; 3.5% to 6.5%; 3.5% to 6%; 3.5% to 5.5%; 3.5% to 5%; 3.5% to 4.5%; 3.5% to 4%; 4% to 11%; 4% to 10.5%; 4% to 10%; 4% to 9.5%; 4% to 9%; 4% to 8.5%; 4% to 8%; 4% to 7.5%; 4% to 7%; 4% to 6.5%; 4% to 6%; 4% to 5.5%; 4% to 5%; 4% to 4.5%; 4.5% to 11%; 4.5% to 10.5%; 4.5% to 10%; 4.5% to 9.5%; 4.5% to 9%; 4.5% to 8.5%; 4.5% to 8%; 4.5% to 7.5%; 4.5% to 7%; 4.5% to 6.5%; 4.5% to 6%; 4.5% to 5.5%; 4.5% to 5%; 5% to 11%; 5% to 10.5%; 5% to 10%; 5% to 9.5%; 5% to 9%; 5% to 8.5%; 5% to 8%; 5% to 7.5%; 5% to 7%; 5% to 6.5%; 5% to 6%; 5% to 5.5%; 5.5% to 11%; 5.5% to 10.5%; 5.5% to 10%; 5.5% to 9.5%; 5.5% to 9%; 5.5% to 8.5%; 5.5% to 8%; 5.5% to 7.5%; 5.5% to 7%; 5.5% to 6.5%; 5.5% to 6%; 6% to 11%; 6% to 10.5%; 6% to 10%; 6% to 9.5%; 6% to 9%; 6% to 8.5%; 6% to 8%; 6% to 7.5%; 6% to 7%; 6% to 6.5%; 6.5% to 11%; 6.5% to 10.5%; 6.5% to 10%; 6.5% to 9.5%; 6.5% to 9%; 6.5% to 8.5%; 6.5% to 8%; 6.5% to 7.5%; 6.5% to 7%; 7% to 11%; 7% to 10.5%; 7% to 10%; 7% to 9.5%; 7% to 9%; 7% to 8.5%; 7% to 8%; 7% to 7.5%; 7.5% to 11%; 7.5% to 10.5%; 7.5% to 10%; 7.5% to 9.5%; 7.5% to 9%; 7.5% to 8.5%; 7.5% to 8%; 8% to 11%; 8% to 10.5%; 8% to 10%; 8% to 9.5%; 8% to 9%; 8% to 8.5%; 8.5% to 11%; 8.5% to 10.5%; 8.5% to 10%; 8.5% to 9.5%; 8.5% to 9%; 8% to 11%; 8% to 10.5%; 8% to 10%; 8% to 9.5%; 8.5% to 11%; 8.5% to 10.5%; 8.5% to 10%; 8.5% to 9%; 9% to 11%; 9% to 10.5%; 9% to 10%; %; 9.5% to 11%; 9.5% to 10.5%; 9.5% to 10%; 10% to 11%; 10% to 10.5%; or 10.5% to 11% (w/v) of a tonicity modifier.

In certain embodiments, the formulation comprises about 3.5%, 4%, 4.5%, 5%, 5.5%, 6%, 6.5%, 7%, 7.5%, 8%, 8.5%, 9%, 9.5%, 10%, 10.5%, or 11% (w/v) of a tonicity modifier. In certain embodiments of the formulation, a tonicity modifier is present in an amount of 3.5%, 4%, 4.5%, 5%, 5.5%, 6%, 6.5%, 7% 7.5%, 8%, 8.5%, 9% 9.5%, 10%, 10.5%, or 11% (w/v). In certain embodiments, the formulation comprises 6.9% or about 6.9% of a tonicity modifier.

In certain embodiments, the tonicity modifier is a disaccharide. In certain embodiments, the tonicity modifier is sucrose.

The formulations may be further characterized according to the pH of the formulation. In certain embodiments, the formulation has a pH in the range of 7.8 to 8.2. In certain embodiments, the formulation has a pH of 8.0. In certain embodiments, the pH is in the range of 7.5 to 8.5; 7.5 to 8.4; 7.5 to 8.3; 7.5 to 8.2; 7.5 to 8.1; 7.5 to 8; 7.5 to 7.9; 7.5 to 7.8; 7.5 to 7.7; 7.5 to 7.6; 7.6 to 8.5; 7.6 to 8.4; 7.6 to 8.3; 7.6 to 8.2; 7.6 to 8.1; 7.6 to 8; 7.6 to 7.9; 7.6 to 7.8; 7.6 to 7.7; 7.7 to 8.5; 7.7 to 8.4; 7.7 to 8.3; 7.7 to 8.2; 7.7 to 8.1; 7.7 to 8; 7.7 to 7.9; 7.7 to 7.8; 7.8 to 8.5; 7.8 to 8.4; 7.8 to 8.3; 7.8 to 8.2; 7.8 to 8.1; 7.8 to 8; 7.8 to 7.9; 7.9 to 8.5; 7.9 to 8.4; 7.9 to 8.3; 7.9 to 8.2; 7.9 to 8.1; 7.9 to 8; 8 to 8.5; 8 to 8.4; 8 to 8.3; 8 to 8.2; 8 to 8.1; 8.1 to 8.5; 8.1 to 8.4; 8.1 to 8.3; 8.1 to 8.2; 8.2 to 8.5; 8.2 to 8.4; 8.2 to 8.3; 8.3 to 8.5; 8.3 to 8.4; or 8.4 to 8.5. In certain embodiments, the formulation has a pH of 7.5, 7.6, 7.7, 7.8, 7.9, 8.0, 8.1, 8.2, 8.3, 8.4, or 8.5.

It may be useful to adjust the pH of the aqueous formulation. Accordingly, in certain embodiments, the formulation comprises one or more pH adjusters. In certain embodiments, the pH adjuster is one or more of an alkali metal hydroxide or hydrochloric acid. In certain embodiments, the pH adjuster is one or more of sodium hydroxide or hydrochloric acid. In certain embodiments, the pH adjuster is sodium hydroxide. In certain embodiments, the pH adjuster is hydrochloric acid.

The formulation may be further characterized according to the amount of water in the formulation. In certain embodiments, the formulation comprises at least 90% or 91% (w/v) water. In certain embodiments, the formulation comprises at least 91% (w/v) water. In certain embodiments, the formulation comprises 90% or 91% (w/v) water.

### Part B—Second Aqueous Formulation

Another aspect of the invention provides an aqueous formulation comprising:
(a) about 0.5% (w/v) of a compound of Formula I, wherein Formula I is represented by:

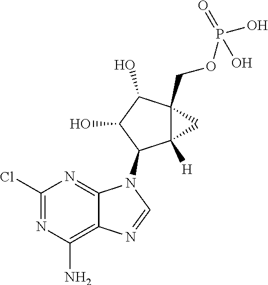 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) about 0.02% (w/v) of a chelating agent selected from ethylenediamine tetraacetic acid, a pharmaceutically acceptable salt of ethylenediamine tetraacetic acid, or a mixture thereof,
(c) about 20 mM of a buffer comprising citric acid and a pharmaceutically acceptable salt of citric acid;
(d) about 6.9% (w/v) of a tonicity modifier; and
(e) at least 90% (w/v) water; and
having a pH of about 8.

The Second Aqueous Formulation may be further described according to additional features, such as the amounts and identities of constituents in the formulation. A more thorough description of such features is provided below. The invention embraces all permutations and combinations of these features.

For example, in certain embodiments, the compound of Formula I is a pharmaceutically acceptable salt of

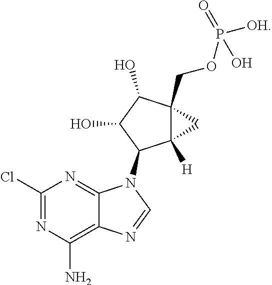 [SMILES: Nc1nc(Cl)nc2c1ncn2[C@H]1[C@H](O)[C@H](O)[C@]2(COP(=O)(O)O)C[C@H]12]

For instance, the compound of Formula I may be an alkali metal or alkaline earth metal pharmaceutically acceptable salt. In certain embodiments, the compound of Formula I is a sodium salt.

The formulation may also comprise about 0.02% (w/v) of a chelating agent. For instance, the chelating agent may be ethylenediamine tetraacetic acid (EDTA), a pharmaceutically acceptable salt of ethylenediamine tetraacetic acid, or a mixture thereof. In certain embodiments, the chelating agent is EDTA. In certain embodiments, the chelating agent is a pharmaceutically acceptable salt of EDTA. In certain embodiments, the chelating agent is a mixture of EDTA and a pharmaceutically acceptable salt of EDTA.

In certain embodiments, the formulation comprises 6.9% of a tonicity modifier. In certain embodiments, the tonicity modifier is a disaccharide. In certain embodiments, the tonicity modifier is sucrose.

In certain embodiments, the formulation comprises at least 91% water.

### Part C—Third Aqueous Formulation

Another aspect of the invention provides an aqueous formulation comprising:
(a) about 0.5% (w/v) of a compound of Formula I, wherein Formula I is represented by:

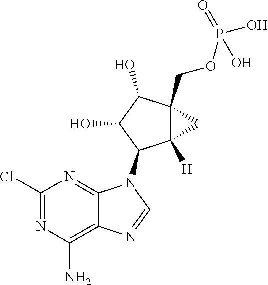 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) about 0.02% (w/v) of a chelating agent selected from ethylenediamine tetraacetic acid, a pharmaceutically acceptable salt of ethylenediamine tetraacetic acid, or a mixture thereof,
(c) about 20 mM of a buffer comprising citric acid and a pharmaceutically acceptable salt of citric acid;
(d) about 6.9% (w/v) sucrose; and
(e) at least 90% (w/v) water; and
having a pH of about 8.

The Third Aqueous Formulation may be further described according to additional features, such as the amounts and identities of constituents in the formulation. A more thorough description of such features is provided below. The invention embraces all permutations and combinations of these features.

For example, in certain embodiments, the formulation comprises 0.02% (w/v) of a chelating agent. For instance, the chelating agent may be ethylenediamine tetraacetic acid (EDTA), a pharmaceutically acceptable salt of ethylenediamine tetraacetic acid, or a mixture thereof. In certain embodiments, the chelating agent is EDTA. In certain embodiments, the chelating agent is a pharmaceutically acceptable salt of EDTA. In certain embodiments, the chelating agent is a mixture of EDTA and a pharmaceutically acceptable salt of EDTA

In certain embodiments, the formulation comprises at least 91% (w/v) water.

In certain embodiments, the formulation has a pH in the range of 7.8 to 8.2. In certain embodiments, the formulation has a pH of 8.0.

In a more specific embodiment, the formulation comprises:
(a) 0.5% (w/v) of a compound of Formula I, wherein Formula I is represented by:

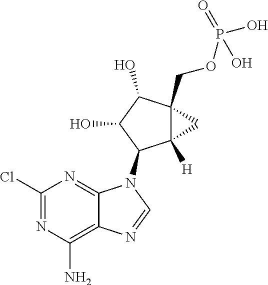 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) 0.02% (w/v) of a chelating agent selected from ethylenediamine tetraacetic acid, a pharmaceutically acceptable salt of ethylenediamine tetraacetic acid, or a mixture thereof;
(c) 20 mM of a buffer comprising citric acid and a pharmaceutically acceptable salt of citric acid;
(d) 6.9% (w/v) sucrose; and
(e) at least 90% (w/v) water; and
having a pH of 8.

### Part D—Fourth Aqueous Formulation

Another aspect of the invention provides an aqueous formulation comprising:
(a) from 0.25% (w/v) to 0.75% (w/v) of a compound of Formula I, wherein Formula I is represented by:

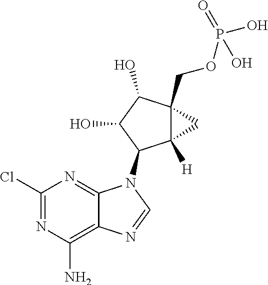 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) a chelating agent; and
(c) at least 85% (w/v) water.

The Fourth Aqueous Formulation may be further described according to additional features, such as the amounts and identities of constituents in the formulation. A more thorough description of such features is provided below. The invention embraces all permutations and combinations of these features.

In a more specific embodiment, the invention provides an aqueous formulation comprising:
(a) from 0.25% (w/v) to 0.75% (w/v) of a compound of Formula I, wherein Formula I is represented by:

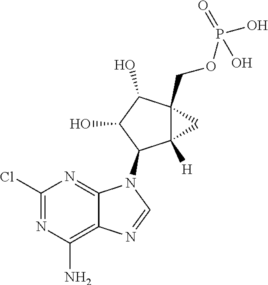 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) from 0.01% (w/v) to 0.06% (w/v) of a chelating agent;
(c) a buffer; and
(d) at least 85% (w/v) water; and
having a pH in the range of 7.0 to 8.5.

In a more specific embodiment, the invention provides an aqueous formulation comprising:
(a) from 0.25% (w/v) to 0.75% (w/v) of a compound of Formula I, wherein Formula I is represented by:

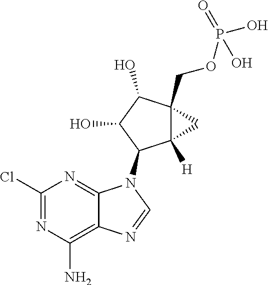 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) from 0.01% (w/v) to 0.06% (w/v) of a chelating agent;
(c) from 0.25% (w/v) to 2% (w/v) of a buffer; and
(d) at least 85% (w/v) water; and
having a pH in the range of 7.0 to 8.5.

For example, in certain embodiments, the compound of Formula I is a pharmaceutically acceptable salt of

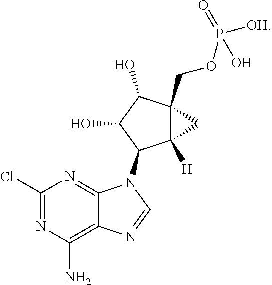 [SMILES: Nc1nc(Cl)nc2c1ncn2[C@H]1[C@H](O)[C@H](O)[C@]2(COP(=O)(O)O)C[C@H]12]

In certain embodiments, the compound of Formula I is an alkali metal or alkaline earth metal pharmaceutically acceptable salt of

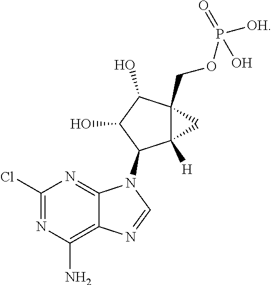 [SMILES: Nc1nc(Cl)nc2c1ncn2[C@H]1[C@H](O)[C@H](O)[C@]2(COP(=O)(O)O)C[C@H]12]

In certain embodiments, the compound of Formula I is a sodium salt of

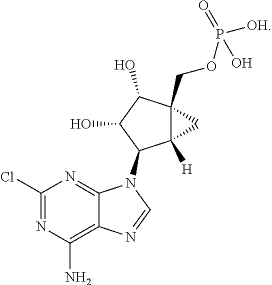 [SMILES: Nc1nc(Cl)nc2c1ncn2[C@H]1[C@H](O)[C@H](O)[C@]2(COP(=O)(O)O)C[C@H]12]

As generally described above, the formulation comprises from 0.25% (w/v) to 0.75% (w/v) of a compound of Formula I. In certain embodiments, the compound of Formula I is present in an amount of from 0.4% (w/v) to 0.6% (w/v). In certain embodiments, the compound of Formula I is present in an amount of about 0.5% (w/v). In certain embodiments, the compound of Formula I is present in an amount of 0.5% (w/v).

In certain embodiments, the formulation comprises from 0.25% to 0.75% (w/v); 0.25% to 0.70% (w/v); 0.25% to 0.65% (w/v); 0.25% to 0.60% (w/v); 0.25% to 0.55% (w/v); 0.25% to 0.50% (w/v); 0.25% to 0.45% (w/v); 0.25% to 0.40% (w/v); 0.25% to 0.35% (w/v); 0.25% to 0.30% (w/v); 0.30% to 0.75% (w/v); 0.30% to 0.70% (w/v); 0.30% to 0.65% (w/v); 0.30% to 0.60% (w/v); 0.30% to 0.55% (w/v); 0.30% to 0.50% (w/v); 0.30% to 0.45% (w/v); 0.30% to 0.40% (w/v); 0.30% to 0.35% (w/v); 0.35% to 0.75% (w/v); 0.35% to 0.70% (w/v); 0.35% to 0.65% (w/v); 0.35% to 0.60% (w/v); 0.35% to 0.55% (w/v); 0.35% to 0.50% (w/v); 0.35% to 0.45% (w/v); 0.35% to 0.40% (w/v); 0.40% to 0.75% (w/v); 0.40% to 0.70% (w/v); 0.40% to 0.65% (w/v); 0.40% to 0.60% (w/v); 0.40% to 0.55% (w/v); 0.40% to 0.50% (w/v); 0.40% to 0.45% (w/v); 0.45% to 0.75% (w/v); 0.45% to 0.70% (w/v); 0.45% to 0.65% (w/v); 0.45% to 0.60% (w/v); 0.45% to 0.55% (w/v); 0.45% to 0.50% (w/v); 0.50% to 0.75% (w/v); 0.50% to 0.70% (w/v); 0.50% to 0.65% (w/v); 0.50% to 0.60% (w/v); 0.50% to 0.55% (w/v); 0.55% to 0.75% (w/v); 0.55% to 0.70% (w/v); 0.55% to 0.65% (w/v); 0.55% to 0.60% (w/v); 0.60% to 0.75% (w/v); 0.60% to 0.70% (w/v); 0.60% to 0.65% (w/v); 0.65% to 0.75% (w/v); 0.65% to 0.70% (w/v); or 0.70% to 0.75% (w/v) of a compound of Formula I.

In certain embodiments, the formulation comprises about 0.25%, 0.30%, 0.35%, 0.40%, 0.45%, 0.50%, 0.55%, 0.60%, 0.65%, 0.70%, or 0.75% (w/v) of a compound of Formula I. In certain embodiments of the formulation, a compound of Formula I is present in an amount of 0.25%, 0.30%, 0.35%, 0.40%, 0.45%, 0.50%, 0.55%, 0.60%, 0.65%, 0.70%, or 0.75% (w/v).

In certain embodiments, the chelating agent is a carboxylic acid or carboxylate salt compound. For instance, in certain embodiments, the chelating agent is ethylene glycol tetraacetic acid, ethylenediamine tetraacetic acid, a C₄₋₁₀ hydroxylalkyl tricarboxylic acid, or a pharmaceutically acceptable salt of any of the foregoing, or a mixture of any of the foregoing. In certain embodiments, the chelating agent is selected from EDTA, a pharmaceutically acceptable salt of EDTA, or a mixture of EDTA and a pharmaceutically acceptable salt of EDTA. In certain embodiments, the chelating agent is selected from ethylenediamine tetraacetic acid, a pharmaceutically acceptable salt of ethylenediamine tetraacetic acid, or a mixture thereof.

In certain embodiments, the formulation comprises from 0.01% (w/v) to 0.03% (w/v) of the chelating agent. In certain embodiments, the formulation comprises about 0.02% (w/v) of the chelating agent. In certain embodiments, the formulation comprises 0.02% (w/v) of the chelating agent.

The formulation may be further characterized according to the buffer. For example, in certain embodiments, the formulation comprises from 10 mM to 30 mM of the buffer. In certain embodiments, the formulation comprises from 15 mM to 25 mM of the buffer. In certain embodiments, the formulation comprises about 20 mM of the buffer. In certain embodiments, the formulation comprises 20 mM of the buffer. In certain embodiments, the formulation comprises about 10, 11, 12, 13, 14, 15, 16, 17, 18, 19, 20, 21, 22, 23, 24, 25, 26, 27, 28, 29 or 30 mM of a buffer. In certain embodiments, the formulation comprises 10, 11, 12, 13, 14, 15, 16, 17, 18, 19, 20, 21, 22, 23, 24, 25, 26, 27, 28, 29 or 30 mM of a buffer.

In certain embodiments, the formulation comprises from 0.25% (w/v) to 2% (w/v) of a buffer. In certain embodiments, the formulation comprises from 0.25% to 2% (w/v); 0.25% to 1.75%; 0.25% to 1.5% (w/v); 0.25% to 1.25%; 0.25% to 1% (w/v); 0.25% to 0.75%; 0.25% to 0.5%; 0.5% to 2% (w/v); 0.5% to 1.75%; 0.5% to 1.5% (w/v); 0.5% to 1.25%; 0.5% to 1% (w/v); 0.5% to 0.75%; 0.75% to 2% (w/v); 0.75% to 1.75%; 0.75% to 1.5% (w/v); 0.75% to 1.25%; 0.75% to 1% (w/v); 1% to 2% (w/v); 1% to 1.75%; 1% to 1.5% (w/v); 1% to 1.25%; 1.25% to 2% (w/v); 1.25% to 1.75%; 1.25% to 1.5% (w/v); 1.5% to 2% (w/v); 1.5% to 1.75%; or 1.75% to 2% (w/v) of a buffer. In certain embodiments, the formulation comprises from 0.4% to 0.6%; 0.4% to 0.5%; or 0.5% to 0.6% (w/v) of a buffer.

In certain embodiments, the formulation comprises about 0.25%, 0.4%, 0.50%, 0.6%, 0.75%, 1%, 1.25%, 1.5%, 1.75% or 2% (w/v) of a buffer. In certain embodiments of the formulation, a buffer is present in an amount of 0.25%, 0.4%, 0.50%, 0.6%, 0.75%, 1%, 1.25%, 1.5%, 1.75% or 2% (w/v).

The buffer may comprise, for instance, a carboxylic acid compound and a carboxylate salt compound. In certain embodiments, the buffer comprises a C₄₋₁₀ hydroxylalkyl dicarboxylic acid, a C₄₋₁₀ hydroxylalkyl tricarboxylic acid, a C₁₋₆ alkanoic acid, glutamic acid, phosphoric acid, or a pharmaceutically acceptable salt of any of the foregoing. In certain embodiments, the buffer comprises a C₄₋₁₀ hydroxylalkyl tricarboxylic acid, or a pharmaceutically acceptable salt thereof. In certain embodiments, the buffer comprises citric acid and a pharmaceutically acceptable salt of citric acid.

In certain embodiments, the formulation further comprises a tonicity modifier. For instance, in certain embodiments, the formulation comprises from 1% to 15% (w/v) of a tonicity modifier. In certain embodiments, the tonicity modifier is present in the formulation in an amount of from 1% (w/v) to 15% (w/v). In certain embodiments, the tonicity modifier is present in the formulation in an amount of from 5% (w/v) to 9% (w/v). In certain embodiments, the tonicity modifier is present in the formulation in an amount of about 6.9% (w/v). In certain embodiments, the tonicity modifier is present in the formulation in an amount of 6.9% (w/v).

In certain embodiments, the formulation comprises from 1% to 15%; 1% to 14%; 1% to 13%; 1% to 12%; 1% to 11%; 1% to 10%; 1% to 9%; 1% to 8%; 1% to 7%; 1% to 6%; 1% to 5%; 1% to 4%; 1% to 3%; 1% to 2%; 2% to 15%; 2% to 14%; 2% to 13%; 2% to 12%; 2% to 12%; 2% to 10%; 2% to 9%; 2% to 8%; 2% to 7%; 2% to 6%; 2% to 5%; 2% to 4%; 2% to 3%; 3% to 15%; 3% to 14%; 3% to 13%; 3% to 12%; 3% to 12%; 3% to 10%; 3% to 9%; 3% to 8%; 3% to 7%; 3% to 6%; 3% to 5%; 3% to 4%; 4% to 15%; 4% to 14%; 4% to 13%; 4% to 12%; 4% to 12%; 4% to 10%; 4% to 9%; 4% to 8%; 4% to 7%; 4% to 6%; 4% to 5%; 5% to 15%; 5% to 14%; 5% to 13%; 5% to 12%; 5% to 12%; 5% to 10%; 5% to 9%; 5% to 8%; 5% to 7%; 5% to 6%; 6% to 15%; 6% to 14%; 6% to 13%; 6% to 12%; 6% to 12%; 6% to 10%; 6% to 9%; 6% to 8%; 6% to 7%; 7% to 15%; 7% to 14%; 7% to 13%; 7% to 12%; 7% to 12%; 7% to 10%; 7% to 9%; 7% to 8%; 8% to 15%; 8% to 14%; 8% to 13%; 8% to 12%; 8% to 12%; 8% to 10%; 8% to 9%; 9% to 15%; 9% to 14%; 9% to 13%; 9% to 12%; 9% to 12%; 9% to 10%; 10% to 15%; 10% to 14%; 10% to 13%; 10% to 12%; 10% to 11%; 11% to 15%; 11% to 14%; 11% to 13%; 11% to 12%; 12% to 15%; 12% to 14%; 12% to 13%; 13% to 15%; 13% to 14%; or 14% to 15% (w/v) of a tonicity modifier.

In certain embodiments, the formulation comprises about 1%, 2%, 3%, 4%, 5%, 6%, 7%, 8%, 9%, 10%, 11%, 12%, 13%, 14%, or 15% (w/v) of a tonicity modifier. In certain embodiments of the formulation, a tonicity modifier is present in an amount of 1%, 2%, 3%, 4%, 5%, 6%, 7%, 8%, 9% 10%, 11%, 12%, 13%, 14%, or 15% (w/v). In certain embodiments, the formulation comprises 6.9% or about 6.9% of a tonicity modifier.

In certain embodiments, the tonicity modifier is a disaccharide, a monosaccharide, sodium chloride, or potassium chloride. In certain embodiments, the tonicity modifier is a disaccharide. In certain embodiments, the tonicity modifier is sucrose.

The formulations may be further characterized according to the pH. In certain embodiments, the formulation has a pH in the range of 7.8 to 8.2. In certain embodiments, the formulation has a pH of about 8.0. In certain embodiments, the formulation has a pH of 8.0. In certain embodiments, the pH is in the range of 7.5 to 8.5; 7.5 to 8.4; 7.5 to 8.3; 7.5 to 8.2; 7.5 to 8.1; 7.5 to 8; 7.5 to 7.9; 7.5 to 7.8; 7.5 to 7.7; 7.5 to 7.6; 7.6 to 8.5; 7.6 to 8.4; 7.6 to 8.3; 7.6 to 8.2; 7.6 to 8.1; 7.6 to 8; 7.6 to 7.9; 7.6 to 7.8; 7.6 to 7.7; 7.7 to 8.5; 7.7 to 8.4; 7.7 to 8.3; 7.7 to 8.2; 7.7 to 8.1; 7.7 to 8; 7.7 to 7.9; 7.7 to 7.8; 7.8 to 8.5; 7.8 to 8.4; 7.8 to 8.3; 7.8 to 8.2; 7.8 to 8.1; 7.8 to 8; 7.8 to 7.9; 7.9 to 8.5; 7.9 to 8.4; 7.9 to 8.3; 7.9 to 8.2; 7.9 to 8.1; 7.9 to 8; 8 to 8.5; 8 to 8.4; 8 to 8.3; 8 to 8.2; 8 to 8.1; 8.1 to 8.5; 8.1 to 8.4; 8.1 to 8.3; 8.1 to 8.2; 8.2 to 8.5; 8.2 to 8.4; 8.2 to 8.3; 8.3 to 8.5; 8.3 to 8.4; or 8.4 to 8.5. In certain embodiments, the formulation has a pH of 7.5, 7.6, 7.7, 7.8, 7.9, 8.0, 8.1, 8.2, 8.3, 8.4, or 8.5.

It may be useful to adjust the pH of the aqueous formulation. Accordingly, in certain embodiments, the formulation comprises one or more pH adjusters. In certain embodiments, the formulation comprises a pH adjuster. In certain embodiments, the pH adjuster is one or more of an alkali metal hydroxide or hydrochloric acid. In certain embodiments, the pH adjuster is one or more of sodium hydroxide or hydrochloric acid. In certain embodiments, the pH adjuster is sodium hydroxide. In certain embodiments, the pH adjuster is hydrochloric acid.

In certain embodiments, the formulation comprises at least 85% (w/v) water. In certain embodiments, the formulation comprises at least 90% (w/v) water. In certain embodiments, the formulation comprises at least 85%, 86%, 87%, 88%, 89%, 90%, 91%, 92%, 93%, 94%, or 95%, (w/v) water. In certain embodiments, the formulation comprises 85%, 86%, 87%, 88%, 89%, 90%, 91%, 92%, 93%, 94%, or 95% (w/v) water.

### Part E—Fifth Aqueous Formulation

Another aspect of the invention provides an aqueous formulation comprising:
(a) a compound of Formula I, wherein Formula I is represented by:

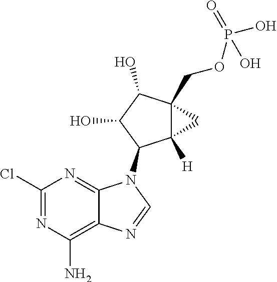 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) a chelating agent; and
(c) water.

The Fifth Aqueous Formulation may be further described according to additional features, such as the amounts and identities of constituents in the formulation. A more thorough description of such features is provided below. The invention embraces all permutations and combinations of these features.

In certain embodiments, the formulation contains from about 6 mM to about 19 mM of a compound of Formula I. In certain embodiments, the formulation contains from about 10 mM to about 16 mM of a compound of Formula I. In certain embodiments, the formulation contains from about 11 mM to about 14 mM of a compound of Formula I. In certain embodiments, the formulation contains from about 12 mM to about 13 mM of a compound of Formula I. In certain embodiments, the formulation contains from 6 mM to 19 mM of a compound of Formula I. In certain embodiments, the formulation contains from 10 mM to 16 mM of a compound of Formula I. In certain embodiments, the formulation contains from 11 mM to 14 mM of a compound of Formula I. In certain embodiments, the formulation contains from 12 mM to 13 mM of a compound of Formula I.

In certain embodiments, the formulation contains about 13 mM of a compound of Formula I. In certain embodiments, the formulation contains 13 mM of a compound of Formula I. In certain embodiments, the formulation contains 12.8 mM of a compound of Formula I.

In certain embodiments, the formulation comprises at least 85% (w/v) water.

In a more specific embodiment, the invention provides an aqueous formulation comprising:
(a) from 6 mM to 19 mM of a compound of Formula I, wherein Formula I is represented by:

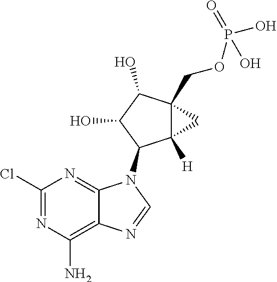 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) a chelating agent; and
(c) at least 85% (w/v) water.

In a more specific embodiment, the invention provides an aqueous formulation comprising:
(a) from 6 mM to 19 mM of a compound of Formula I, wherein Formula I is represented by:

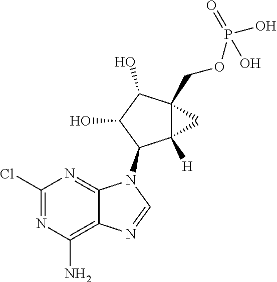 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) from 0.01% (w/v) to 0.06% (w/v) of a chelating agent;
(c) from 0.25% (w/v) to 2% (w/v) of a buffer; and
(d) at least 85% (w/v) water; and
having a pH in the range of 7.0 to 8.5.

In a more specific embodiment, the invention provides an aqueous formulation comprising:
(a) from 11 mM to 16 mM of a compound of Formula I, wherein Formula I is represented by:

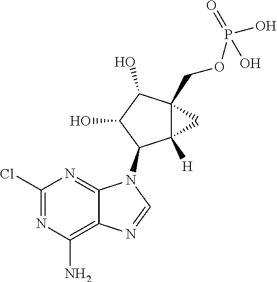 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) from 0.01% (w/v) to 0.06% (w/v) of a chelating agent;
(c) from 0.25% (w/v) to 2% (w/v) of a buffer; and
(d) at least 85% (w/v) water; and
having a pH in the range of 7.0 to 8.5.

In a more specific embodiment, the invention provides an aqueous formulation comprising:
(a) from 12 mM to 14 mM of a compound of Formula I, wherein Formula I is represented by:

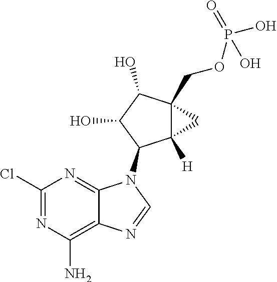 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) from 0.01% (w/v) to 0.06% (w/v) of a chelating agent;
(c) from 0.25% (w/v) to 2% (w/v) of a buffer; and
(d) at least 85% (w/v) water; and
having a pH in the range of 7.0 to 8.5.

In a more specific embodiment, the invention provides an aqueous formulation comprising:
(a) from 12 mM to 13 mM of a compound of Formula I, wherein Formula I is represented by:

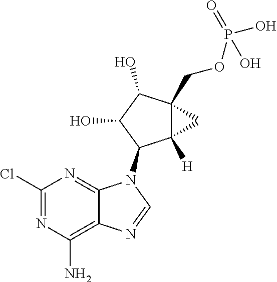 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) from 0.01% (w/v) to 0.06% (w/v) of a chelating agent;
(c) from 0.25% (w/v) to 2% (w/v) of a buffer; and
(d) at least 85% (w/v) water; and
having a pH in the range of 7.0 to 8.5.

In certain embodiments, the compound of Formula I is a pharmaceutically acceptable salt of

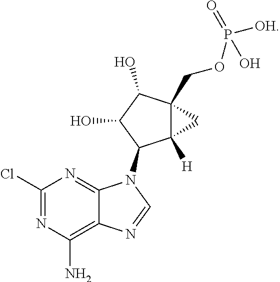 [SMILES: Nc1nc(Cl)nc2c1ncn2[C@H]1[C@H](O)[C@H](O)[C@]2(COP(=O)(O)O)C[C@H]12]

In certain embodiments, the compound of Formula I is an alkali metal or alkaline earth metal pharmaceutically acceptable salt of

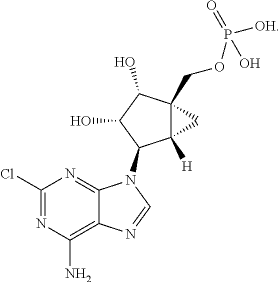 [SMILES: Nc1nc(Cl)nc2c1ncn2[C@H]1[C@H](O)[C@H](O)[C@]2(COP(=O)(O)O)C[C@H]12]

In certain embodiments, the compound of Formula I is a sodium salt of

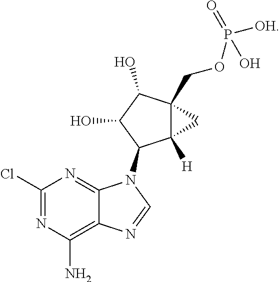 [SMILES: Nc1nc(Cl)nc2c1ncn2[C@H]1[C@H](O)[C@H](O)[C@]2(COP(=O)(O)O)C[C@H]12]

In certain embodiments, the chelating agent is a carboxylic acid or carboxylate salt compound. For instance, in certain embodiments, the chelating agent is ethylene glycol tetraacetic acid, ethylenediamine tetraacetic acid, a C₄₋₁₀ hydroxylalkyl tricarboxylic acid, or a pharmaceutically acceptable salt of any of the foregoing, or a mixture of any of the foregoing. In certain embodiments, the chelating agent is selected from EDTA, a pharmaceutically acceptable salt of EDTA, or a mixture of EDTA and a pharmaceutically acceptable salt of EDTA.

In certain embodiments, the formulation comprises from 0.01% (w/v) to 0.03% (w/v) of the chelating agent. In certain embodiments, the formulation comprises about 0.02% (w/v) of the chelating agent. In certain embodiments, the formulation comprises 0.02% (w/v) of the chelating agent.

The formulation may be further characterized according to the buffer. For example, in certain embodiments, the formulation comprises from 10 mM to 30 mM of the buffer. In certain embodiments, the formulation comprises from 15 mM to 25 mM of the buffer. In certain embodiments, the formulation comprises about 20 mM of the buffer. In certain embodiments, the formulation comprises 20 mM of the buffer. In certain embodiments, the formulation comprises about 10, 11, 12, 13, 14, 15, 16, 17, 18, 19, 20, 21, 22, 23, 24, 25, 26, 27, 28, 29 or 30 mM of a buffer. In certain embodiments, the formulation comprises 10, 11, 12, 13, 14, 15, 16, 17, 18, 19, 20, 21, 22, 23, 24, 25, 26, 27, 28, 29 or 30 mM of a buffer.

In certain embodiments, the formulation comprises from 0.25% (w/v) to 2% (w/v) of a buffer. In certain embodiments, the formulation comprises from 0.25% to 2% (w/v); 0.25% to 1.75%; 0.25% to 1.5% (w/v); 0.25% to 1.25%; 0.25% to 1% (w/v); 0.25% to 0.75%; 0.25% to 0.5%; 0.5% to 2% (w/v); 0.5% to 1.75%; 0.5% to 1.5% (w/v); 0.5% to 1.25%; 0.5% to 1% (w/v); 0.5% to 0.75%; 0.75% to 2% (w/v); 0.75% to 1.75%; 0.75% to 1.5% (w/v); 0.75% to 1.25%; 0.75% to 1% (w/v); 1% to 2% (w/v); 1% to 1.75%; 1% to 1.5% (w/v); 1% to 1.25%; 1.25% to 2% (w/v); 1.25% to 1.75%; 1.25% to 1.5% (w/v); 1.5% to 2% (w/v); 1.5% to 1.75%; or 1.75% to 2% (w/v) of a buffer. In certain embodiments, the formulation comprises from 0.4% to 0.6%; 0.4% to 0.5%; or 0.5% to 0.6% (w/v) of a buffer.

In certain embodiments, the formulation comprises about 0.25%, 0.4%, 0.50%, 0.6%, 0.75%, 1%, 1.25%, 1.5%, 1.75% or 2% (w/v) of a buffer. In certain embodiments of the formulation, a buffer is present in an amount of 0.25%, 0.4%, 0.50%, 0.6%, 0.75%, 1%, 1.25%, 1.5%, 1.75% or 2% (w/v).

The buffer may comprise, for instance, a carboxylic acid compound and a carboxylate salt compound. In certain embodiments, the buffer comprises a C₄₋₁₀ hydroxylalkyl dicarboxylic acid, a C₄₋₁₀ hydroxylalkyl tricarboxylic acid, a C₁₋₆ alkanoic acid, glutamic acid, phosphoric acid, or a pharmaceutically acceptable salt of any of the foregoing. In certain embodiments, the buffer comprises a C₄₋₁₀ hydroxylalkyl tricarboxylic acid, or a pharmaceutically acceptable salt thereof. In certain embodiments, the buffer comprises citric acid and a pharmaceutically acceptable salt of citric acid.

In certain embodiments, the formulation further comprises a tonicity modifier. For instance, in certain embodiments, the formulation comprises from 1% to 15% (w/v) of a tonicity modifier. In certain embodiments, the tonicity modifier is present in the formulation in an amount of from 1% (w/v) to 15% (w/v). In certain embodiments, the tonicity modifier is present in the formulation in an amount of from 5% (w/v) to 9% (w/v). In certain embodiments, the tonicity modifier is present in the formulation in an amount of about 6.9% (w/v). In certain embodiments, the tonicity modifier is present in the formulation in an amount of 6.9% (w/v).

In certain embodiments, the formulation comprises from 1% to 15%; 1% to 14%; 1% to 13%; 1% to 12%; 1% to 11%; 1% to 10%; 1% to 9%; 1% to 8%; 1% to 7%; 1% to 6%; 1% to 5%; 1% to 4%; 1% to 3%; 1% to 2%; 2% to 15%; 2% to 14%; 2% to 13%; 2% to 12%; 2% to 12%; 2% to 10%; 2% to 9%; 2% to 8%; 2% to 7%; 2% to 6%; 2% to 5%; 2% to 4%; 2% to 3%; 3% to 15%; 3% to 14%; 3% to 13%; 3% to 12%; 3% to 12%; 3% to 10%; 3% to 9%; 3% to 8%; 3% to 7%; 3% to 6%; 3% to 5%; 3% to 4%; 4% to 15%; 4% to 14%; 4% to 13%; 4% to 12%; 4% to 12%; 4% to 10%; 4% to 9%; 4% to 8%; 4% to 7%; 4% to 6%; 4% to 5%; 5% to 15%; 5% to 14%; 5% to 13%; 5% to 12%; 5% to 12%; 5% to 10%; 5% to 9%; 5% to 8%; 5% to 7%; 5% to 6%; 6% to 15%; 6% to 14%; 6% to 13%; 6% to 12%; 6% to 12%; 6% to 10%; 6% to 9%; 6% to 8%; 6% to 7%; 7% to 15%; 7% to 14%; 7% to 13%; 7% to 12%; 7% to 12%; 7% to 10%; 7% to 9%; 7% to 8%; 8% to 15%; 8% to 14%; 8% to 13%; 8% to 12%; 8% to 12%; 8% to 10%; 8% to 9%; 9% to 15%; 9% to 14%; 9% to 13%; 9% to 12%; 9% to 12%; 9% to 10%; 10% to 15%; 10% to 14%; 10% to 13%; 10% to 12%; 10% to 11%; 11% to 15%; 11% to 14%; 11% to 13%; 11% to 12%; 12% to 15%; 12% to 14%; 12% to 13%; 13% to 15%; 13% to 14%; or 14% to 15% (w/v) of a tonicity modifier.

In certain embodiments, the formulation comprises about 1%, 2%, 3%, 4%, 5%, 6%, 7%, 8%, 9%, 10%, 11%, 12%, 13%, 14%, or 15% (w/v) of a tonicity modifier. In certain embodiments of the formulation, a tonicity modifier is present in an amount of 1%, 2%, 3%, 4%, 5%, 6%, 7%, 8%, 9%, 10%, 11%, 12%, 13%, 14%, or 15% (w/v). In certain embodiments, the formulation comprises 6.9% or about 6.9% of a tonicity modifier.

In certain embodiments, the tonicity modifier is a disaccharide, a monosaccharide, sodium chloride, or potassium chloride. In certain embodiments, the tonicity modifier is a disaccharide. In certain embodiments, the tonicity modifier is sucrose.

The formulations may be further characterized according to the pH. In certain embodiments, the formulation has a pH in the range of 7.8 to 8.2. In certain embodiments, the formulation has a pH of about 8.0. In certain embodiments, the formulation has a pH of 8.0. In certain embodiments, the pH is in the range of 7.5 to 8.5; 7.5 to 8.4; 7.5 to 8.3; 7.5 to 8.2; 7.5 to 8.1; 7.5 to 8; 7.5 to 7.9; 7.5 to 7.8; 7.5 to 7.7; 7.5 to 7.6; 7.6 to 8.5; 7.6 to 8.4; 7.6 to 8.3; 7.6 to 8.2; 7.6 to 8.1; 7.6 to 8; 7.6 to 7.9; 7.6 to 7.8; 7.6 to 7.7; 7.7 to 8.5; 7.7 to 8.4; 7.7 to 8.3; 7.7 to 8.2; 7.7 to 8.1; 7.7 to 8; 7.7 to 7.9; 7.7 to 7.8; 7.8 to 8.5; 7.8 to 8.4; 7.8 to 8.3; 7.8 to 8.2; 7.8 to 8.1; 7.8 to 8; 7.8 to 7.9; 7.9 to 8.5; 7.9 to 8.4; 7.9 to 8.3; 7.9 to 8.2; 7.9 to 8.1; 7.9 to 8; 8 to 8.5; 8 to 8.4; 8 to 8.3; 8 to 8.2; 8 to 8.1; 8.1 to 8.5; 8.1 to 8.4; 8.1 to 8.3; 8.1 to 8.2; 8.2 to 8.5; 8.2 to 8.4; 8.2 to 8.3; 8.3 to 8.5; 8.3 to 8.4; or 8.4 to 8.5. In certain embodiments, the formulation has a pH of 7.5, 7.6, 7.7, 7.8, 7.9, 8.0, 8.1, 8.2, 8.3, 8.4, or 8.5.

It may be useful to adjust the pH of the aqueous formulation. Accordingly, in certain embodiments, the formulation comprises one or more pH adjusters. In certain embodiments, the formulation comprises a pH adjuster. In certain embodiments, the pH adjuster is one or more of an alkali metal hydroxide or hydrochloric acid. In certain embodiments, the pH adjuster is one or more of sodium hydroxide or hydrochloric acid. In certain embodiments, the pH adjuster is sodium hydroxide. In certain embodiments, the pH adjuster is hydrochloric acid.

In certain embodiments, the formulation comprises at least 85% (w/v) water. In certain embodiments, the formulation comprises at least 90% (w/v) water. In certain embodiments, the formulation comprises at least 85%, 86%, 87%, 88%, 89%, 90%, 91%, 92%, 93%, 94%, or 95%, (w/v) water. In certain embodiments, the formulation comprises 85%, 86%, 87%, 88%, 89%, 90%, 91%, 92%, 93%, 94%, or 95% (w/v) water.

In a more specific embodiment, the invention provides an aqueous formulation comprising:
(a) from 6 mM to 19 mM of a compound of Formula I, wherein Formula I is represented by:

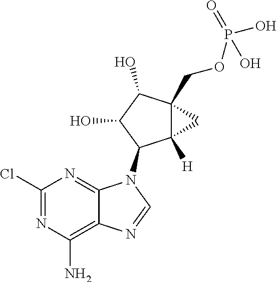 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) from 0.01% (w/v) to 0.03% (w/v) of a chelating agent selected from ethylenediamine tetraacetic acid, a pharmaceutically acceptable salt of ethylenediamine tetraacetic acid, or a mixture thereof;
(c) from 10 mM to 30 mM of a buffer comprising citric acid and a pharmaceutically acceptable salt of citric acid;
(d) from 3.5% (w/v) to 11% (w/v) of a tonicity modifier; and
(e) at least 90% (w/v) water; and
having a pH in the range of 7.5 to 8.5.

In a more specific embodiment, the invention provides an aqueous formulation comprising:
(a) about 12.8 mM of a compound of Formula I, wherein Formula I is represented by:

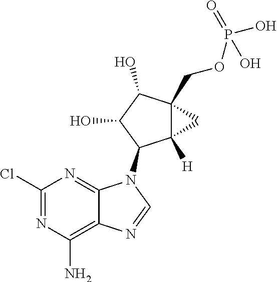 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) about 0.02% (w/v) of a chelating agent selected from ethylenediamine tetraacetic acid, a pharmaceutically acceptable salt of ethylenediamine tetraacetic acid, or a mixture thereof,
(c) about 20 mM of a buffer comprising citric acid and a pharmaceutically acceptable salt of citric acid;
(d) about 6.9% (w/v) of a tonicity modifier; and
(e) at least 90% (w/v) water; and
having a pH of about 8.

In a more specific embodiment, the invention provides an aqueous formulation comprising:
(a) about 12.8 mM of a compound of Formula I, wherein Formula I is represented by:

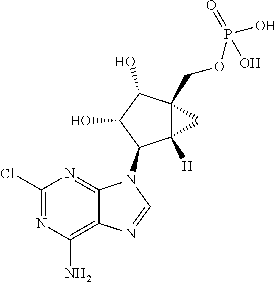 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) about 0.02% (w/v) of a chelating agent selected from ethylenediamine tetraacetic acid, a pharmaceutically acceptable salt of ethylenediamine tetraacetic acid, or a mixture thereof,
(c) about 20 mM of a buffer comprising citric acid and a pharmaceutically acceptable salt of citric acid;
(d) about 6.9% (w/v) sucrose; and
(e) at least 90% (w/v) water; and
having a pH of about 8.

In a more specific embodiment, the invention provides an aqueous formulation comprising:
(a) 12.8 mM of a compound of Formula I, wherein Formula I is represented by:

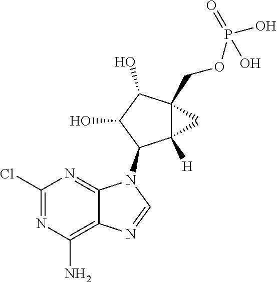 [SMILES: Nc1nc(Cl)nc2c1ncn2[C@H]1[C@H](O)[C@H](O)[C@]2(COP(=O)(O)O)C[C@H]12]

or a pharmaceutically acceptable salt thereof,
(b) 0.02% (w/v) of a chelating agent selected from ethylenediamine tetraacetic acid, a pharmaceutically acceptable salt of ethylenediamine tetraacetic acid, or a mixture thereof;
(c) 20 mM of a buffer comprising citric acid and a pharmaceutically acceptable salt of citric acid;
(d) 6.9% (w/v) sucrose; and
(e) at least 90% (w/v) water; and
having a pH of 8.

### Part F—Additional Features of the First, Second, Third, Fourth, and Fifth Aqueous Formulations

The First, Second, Third, Fourth, and Fifth Aqueous Formulations may be further characterized according to additional features, such as the osmolality, purity upon storage, and other features. A more thorough description of such features is provided below. The invention embraces all permutations and combinations of these features.

For example, in certain embodiments, the formulation has an osmolality in the range of from about 240 to about 340 mOsm/kg. In certain embodiments, the formulation has an osmolality in the range from about 270 mOsm/kg to about 330 mOsm/kg. In certain embodiments, the formulation has an osmolality in the range from about 240 to about 340; about 240 to about 330; about 240 to about 320; about 240 to about 310; about 240 to about 300; about 240 to about 290; about 240 to about 280; about 240 to about 270; about 240 to about 260; about 240 to about 250; about 250 to about 340; about 250 to about 330; about 250 to about 320; about 250 to about 310; about 250 to about 300; about 250 to about 290; about 250 to about 280; about 250 to about 270; about 250 to about 260; about 260 to about 340; about 260 to about 330; about 260 to about 320; about 260 to about 310; about 260 to about 300; about 260 to about 290; about 260 to about 280; about 260 to about 270; about 270 to about 340; about 270 to about 330; about 270 to about 320; about 270 to about 310; about 270 to about 300; about 270 to about 290; about 270 to about 280; about 280 to about 340; about 280 to about 330; about 280 to about 320; about 280 to about 310; about 280 to about 300; about 280 to about 290; about 290 to about 340; about 290 to about 330; about 290 to about 320; about 290 to about 310; about 290 to about 300; about 300 to about 340; about 300 to about 330; about 300 to about 320; about 300 to about 310; about 310 to about 340; about 310 to about 330; about 310 to about 320; about 320 to about 340; about 320 to about 330; or about 330 to about 340 mOsm/kg.

In certain embodiments, the formulation has an osmolality of about 240, 250, 260, 270, 280, 290, 300, 310, 320, 330, or 340 mOsm/kg. In certain embodiments, the formulation has an osmolality of 240, 250, 260, 270, 280, 290, 300, 310, 320, 330, or 340 mOsm/kg.

In certain embodiments of the formulations described herein, less than 0.5%, 1%, 1.5%, or 2% by weight of the compound of Formula I degrades upon storage of the formulation for 4 weeks or greater. In certain embodiments, less than 0.5%, 1%, 1.5%, or 2% by weight of the compound of Formula I degrades upon storage of the formulation for 24 weeks or greater.

In certain embodiments, less than 2% by weight of compound of Formula I degrades upon storage of the formulation at 25° C. for 4 weeks. In certain embodiments, less than 1% by weight of compound of Formula I degrades upon storage of the formulation at 25° C. for 4 weeks. In certain embodiments, less than 0.5% by weight of compound of Formula I degrades upon storage of the formulation at 25° C. for 4 weeks. In certain embodiments of the formulations described herein, less than 0.5%, 1%, 1.5%, or 2% by weight of the compound of Formula I degrades upon storage of the formulation for 4 weeks or greater at 25° C.

In certain embodiments, less than 2% by weight of compound of Formula I degrades upon storage of the formulation at 25° C. for 24 weeks. In certain embodiments, less than 1% by weight of compound of Formula I degrades upon storage of the formulation at 25° C. for 24 weeks. In certain embodiments, less than 0.5% by weight of compound of Formula I degrades upon storage of the formulation at 25° C. for 24 weeks. In certain embodiments, less than 0.5%, 1%, 1.5%, or 2% by weight of the compound of Formula I degrades upon storage of the formulation for 24 weeks or greater at 25° C.

In certain embodiments, less than 2% by weight of compound of Formula I degrades upon storage of the formulation at 40° C. for 4 weeks. In certain embodiments, less than 1% by weight of compound of Formula I degrades upon storage of the formulation at 40° C. for 4 weeks. In certain embodiments, less than 0.5% by weight of compound of Formula I degrades upon storage of the formulation at 40° C. for 4 weeks. In certain embodiments of the formulations described herein, less than 0.5%, 1%, 1.5%, or 2% by weight of the compound of Formula I degrades upon storage of the formulation for 4 weeks or greater at 40° C.

In certain embodiments, less than 2% by weight of compound of Formula I degrades upon storage of the formulation at 40° C. for 24 weeks. In certain embodiments, less than 1% by weight of compound of Formula I degrades upon storage of the formulation at 40° C. for 24 weeks. In certain embodiments, less than 0.5% by weight of compound of Formula I degrades upon storage of the formulation at 40° C. for 24 weeks. In certain embodiments, less than 0.5%, 1%, 1.5%, or 2% by weight of the compound of Formula I degrades upon storage of the formulation for 24 weeks or greater at 40° C.

In certain embodiments, the formulation contains less than 0.02% (w/v) of the following compound or a pharmaceutically acceptable salt thereof:

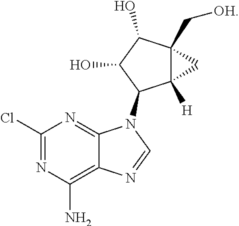 [SMILES: Nc1nc(Cl)nc2c1ncn2[C@H]1[C@H](O)[C@H](O)[C@]2(CO)C[C@H]12]

In certain embodiments, the formulation contains less than 0.01% (w/v) of the following compound or a pharmaceutically acceptable salt thereof:

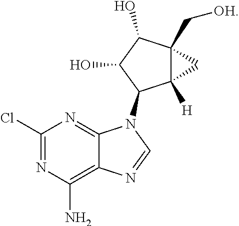 [SMILES: Nc1nc(Cl)nc2c1ncn2[C@H]1[C@H](O)[C@H](O)[C@]2(CO)C[C@H]12]

In certain embodiments, the formulation contains less than 0.02% (w/v) of the following compound or a pharmaceutically acceptable salt thereof upon storage of the formulation at 25° C. for 4 weeks:

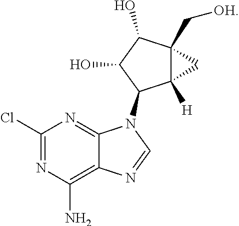 [SMILES: Nc1nc(Cl)nc2c1ncn2[C@H]1[C@H](O)[C@H](O)[C@]2(CO)C[C@H]12]

In certain embodiments, the formulation contains less than 0.02% (w/v) of the following compound or a pharmaceutically acceptable salt thereof upon storage of the formulation at 25° C. for 24 weeks:

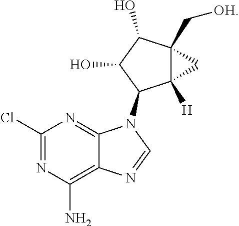 [SMILES: Nc1nc(Cl)nc2c1ncn2[C@H]1[C@H](O)[C@H](O)[C@]2(CO)C[C@H]12]

In certain embodiments, the formulation does not contain a fatty acid ester of polyethoxy sorbitan. In certain embodiments, the formulation does not contain a polysorbate. In certain embodiments, the formulation contains less than 0.01% (w/v) of a fatty acid ester of polyethoxy sorbitan. In certain embodiments, the formulation contains less than 0.01% (w/v) of a polysorbate.

In certain embodiments, the pharmaceutically acceptable salt of citric acid is an alkali metal or alkaline earth metal salt of citric acid. In certain embodiments, the pharmaceutically acceptable salt of citric acid is an alkali metal salt of citric acid. In certain embodiments, the pharmaceutically acceptable salt of citric acid is a sodium salt of citric acid. In certain embodiments, the pharmaceutically acceptable salt of citric acid is an ammonium salt of citric acid.

### II. Aqueous Injectable Formulations

Another aspect of the invention provides an aqueous injectable formulation comprising an aqueous formulation described herein (e.g., an aqueous formulation described in Section I) and a diluent for intravenous administration. In certain embodiments, the diluent for intravenous administration is saline solution. In certain embodiments, the diluent for intravenous administration is Lactated Ringer's solution. In certain embodiments, the aqueous injectable formulation comprises from about 0.3% (w/v) to about 0.5% (w/v) of said compound of Formula I. In certain embodiments, the aqueous injectable formulation comprises about 0.4% (w/v) of said compound of Formula I. In certain embodiments, the aqueous injectable formulation comprises 0.4% (w/v) of said compound of Formula I. In certain embodiments, the aqueous injectable formulation comprises from about 0.01% (w/v) to about 0.1% (w/v) of said compound of Formula I. In certain embodiments, the aqueous injectable formulation comprises from about 0.03% (w/v) to about 0.05% (w/v) of said compound of Formula I. In certain embodiments, the aqueous injectable formulation comprises about 0.04% (w/v) of said compound of Formula I. In certain embodiments, the aqueous injectable formulation comprises 0.04% (w/v) of said compound of Formula I.

Another aspect of the invention provides an aqueous injectable formulation produced by admixing an aqueous formulation described herein (e.g., an aqueous formulation described in Section I) and a diluent for intravenous administration. In certain embodiments, the diluent for intravenous administration is saline solution. In certain embodiments, the diluent for intravenous administration is Lactated Ringer's solution. In certain embodiments, the aqueous injectable formulation comprises from about 0.3% (w/v) to about 0.5% (w/v) of said compound of Formula I. In certain embodiments, the aqueous injectable formulation comprises about 0.4% (w/v) of said compound of Formula I. In certain embodiments, the aqueous injectable formulation comprises 0.4% (w/v) of said compound of Formula I. In certain embodiments, the aqueous injectable formulation comprises from about 0.01% (w/v) to about 0.1% (w/v) of said compound of Formula I. In certain embodiments, the aqueous injectable formulation comprises from about 0.03% (w/v) to about 0.05% (w/v) of said compound of Formula I. In certain embodiments, the aqueous injectable formulation comprises about 0.04% (w/v) of said compound of Formula I. In certain embodiments, the aqueous injectable formulation comprises 0.04% (w/v) of said compound of Formula I.

Another aspect of the invention provides a method for preparing an aqueous injectable formulation, comprising admixing an aqueous formulation described herein (e.g., an aqueous formulation described in Section I) and a diluent for intravenous administration. In certain embodiments, the diluent for intravenous administration is saline solution. In certain embodiments, the diluent for intravenous administration is Lactated Ringer's solution. In certain embodiments, the aqueous injectable formulation comprises from about 0.3% (w/v) to about 0.5% (w/v) of said compound of Formula I. In certain embodiments, the aqueous injectable formulation comprises about 0.4% (w/v) of said compound of Formula I. In certain embodiments, the aqueous injectable formulation comprises 0.4% (w/v) of said compound of Formula I. In certain embodiments, the aqueous injectable formulation comprises from about 0.01% (w/v) to about 0.1% (w/v) of said compound of Formula I. In certain embodiments, the aqueous injectable formulation comprises from about 0.03% (w/v) to about 0.05% (w/v) of said compound of Formula I. In certain embodiments, the aqueous injectable formulation comprises about 0.04% (w/v) of said compound of Formula I. In certain embodiments, the aqueous injectable formulation comprises 0.04% (w/v) of said compound of Formula I.

### III. Therapeutic Applications

The aqueous formulations described herein provide therapeutic benefits to subjects suffering from medical disorders, including cardiac disorders. Accordingly, provided herein is a method for treating a cardiac disorder, comprising administering to a subject in need thereof a therapeutically effective amount of an aqueous injectable formulation described herein (e.g., an aqueous injectable formulation described in Section II), to treat the cardiac disorder.

Another aspect of the invention provides a method for improving cardiac contractile performance in a subject, wherein the method comprises administering to a subject in need thereof an effective amount of an aqueous injectable formulation described herein to improve cardiac contractile performance.

Another aspect of the invention provides a method for improving cardiac function in a subject, wherein the method comprises administering to a subject in need thereof an effective amount of an aqueous injectable formulation described herein to improve cardiac function.

In certain embodiments, the improved cardiac function is characterized by one or more of improved ability of the heart to relax, favorable remodeling in a subject with heart failure, decreased fibrosis, decreased hypertrophy of cardiac myocytes, or improved calcium handling in a myocyte in a heart failure subject.

Another aspect of the invention provides a method of stimulating the activity of a cardiac P2X receptor in a subject, comprising administering to the subject in need thereof an effective amount of an aqueous injectable formulation described herein to stimulate the activity of said cardiac P2X receptor.

In certain embodiments, the subject has a cardiac disorder. In certain embodiments, the cardiac disorder is heart failure, cardiac hypertrophy, ischemic cardiomyopathy, non-ischemic cardiomyopathy, or adverse remodeling and injury following ischemia/reperfusion injury. In certain embodiments, the cardiac disorder is heart failure. In certain embodiments, the cardiac disorder is cardiac hypertrophy. In certain embodiments, the cardiac disorder is ischemic cardiomyopathy. In certain embodiments, the cardiac disorder is non-ischemic cardiomyopathy. In certain embodiments, the cardiac disorder is adverse remodeling and injury following ischemia/reperfusion injury. In certain embodiments, the subject has more than one of any of the foregoing cardiac disorders.

In certain embodiments, the cardiac disorder is heart failure. In certain embodiments, the heart failure is one or more of systolic heart failure or diastolic heart failure. In certain embodiments, the heart failure is systolic heart failure. In certain embodiments, the heart failure is diastolic heart failure.

In certain embodiments, the subject is a human. In certain embodiments, the subject is an adult human. In certain embodiments, the subject is a pediatric human. In certain embodiments, the subject is a geriatric human.

Another aspect of the invention provides for the use of an aqueous formulation described herein in the manufacture of a medicament. In certain embodiments, the medicament is for treating a disease or condition described herein, such as heart failure.

Another aspect of the invention provides for the use of an aqueous formulation described herein for treating a disease or condition, such as a disease or condition described herein, such as heart failure.

Another aspect of this invention is a kit comprising an aqueous formulation described herein. In certain embodiments, the kit further comprises instructions, such as instructions for treating a disease or condition described herein.

### IV. Administration Aspects

The aqueous injectable formulations described herein of the present invention are desirably administered by intravenous administration. Actual dosage amount of the aqueous injectable formulations of this invention may be varied so as to provide an amount of the active ingredient which is effective to achieve the desired therapeutic response for a particular patient, composition, and mode of administration, without being toxic to the patient.

The selected dosage level will depend upon a variety of factors including the activity of the particular aqueous injectable formulations of the present disclosure employed, the route of administration, the time of administration, the rate of excretion or metabolism of the particular compound being employed, the rate and extent of absorption, the duration of the treatment, other drugs, compounds and/or materials used in combination with the particular Aqueous formulation employed, the age, sex, weight, condition, general health and prior medical history of the patient being treated, and like factors well known in the medical arts.

A physician or veterinarian having ordinary skill in the art can readily determine and prescribe the effective amount of the aqueous injectable formulations required. For example, the physician or veterinarian could start doses of the aqueous injectable formulations of the invention at levels lower than that required in order to achieve the desired therapeutic effect and gradually increase the dosage until the desired effect is achieved.

In general, a suitable daily dose of aqueous injectable formulations of the invention will be that amount of the aqueous injectable formulations which is the lowest dose effective to produce a therapeutic effect. Such an effective dose will generally depend upon the factors described above.

If desired, the effective daily dose of the aqueous injectable formulations may be administered as two, three, four, five, six or more sub-doses administered separately at appropriate intervals throughout the day, optionally, in unit dosage forms.

### EXAMPLES

The invention now being generally described, will be more readily understood by reference to the following examples, which are included merely for purposes of illustration of certain aspects and embodiments of the present invention, and are not intended to limit the invention. Starting materials described herein can be obtained from commercial sources or may be readily prepared from commercially available materials using transformations known to those of skill in the art.

### Example 1—Stability Analysis of Aqueous Formulations Containing Compound 1 and Optionally Histidine or Polyethylene Glycol

Compound 1 was formulated into the aqueous formulations set forth in Table 1 below, and the formulations were evaluated for stability to storage for a duration of one week at a temperature of either 4° C., 25° C., or 40° C. Compound 1 has the following formula:

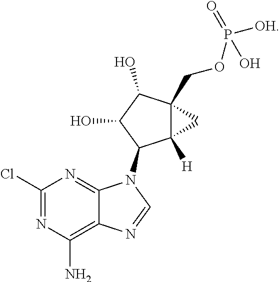 [SMILES: Nc1nc(Cl)nc2c1ncn2[C@H]1[C@H](O)[C@H](O)[C@]2(COP(=O)(O)O)C[C@H]12]

TABLE 1
Aqueous Formulations

| Formulation | |
|---|---|
| No. | Description of the Formulation |
| 1 | Aqueous formulation containing: |
| | Compound 1 at a target concentration of 5 mg/mL |
| | (where the target concentration is expressed |
| | according to the amount of Compound 1 initially |
| | added to the formulation without regard to any |
| | conversion of Compound 1 to a salt form upon |
| | reaction with histidine or other basic species |
| | in the formulation). |
| | 20 mM histidine |
| | at pH 7.4 |
| 2 | Aqueous formulation containing: |
| | Compound 1 at a target concentration of 10 mg/mL |
| | (where the target concentration is expressed |
| | according to the amount of Compound 1 initially |
| | added to the formulation without regard to any |
| | conversion of Compound 1 to a salt form upon |
| | reaction with histidine or other basic species |
| | in the formulation). |
| | 20 mM histidine |
| | at pH 7.4 |
| 3 | Aqueous formulation containing: |
| | Compound 1 at a target concentration of 20 mg/mL |
| | (where the target concentration is expressed |
| | according to the amount of Compound 1 initially |
| | added to the formulation without regard to any |
| | conversion of Compound 1 to a salt form upon |
| | reaction with histidine or other basic species |
| | in the formulation). |
| | 20 mM histidine |
| | pH 7.4 |
| 4 | Aqueous formulation containing: |
| | Compound 1 at a target concentration of 20 mg/mL |
| | (where the target concentration is expressed |
| | according to the amount of Compound 1 initially |
| | added to the formulation without regard to any |
| | conversion of Compound 1 to a salt form upon |
| | reaction with phosphate buffer or other basic species |
| | in the formulation). |
| | 10% polyethylene glycol 300 |
| | Phosphate buffer |
| | at pH 7.4 |
| 5 | Aqueous formulation containing: |
| | Compound 1 at a target concentration of 20 mg/mL |
| | (where the target concentration is expressed |
| | according to the amount of Compound 1 initially |
| | added to the formulation without regard to any |
| | conversion of Compound 1 to a salt form upon |
| | reaction with phosphate buffer or other basic species |
| | in the formulation). |
| | 10% polyethylene glycol 300 |
| | at pH 7.4, where sodium hydroxide was added to the |
| | formulation to achieve a pH of 7.4 for the formulation. |
| 6 | Aqueous formulation containing: |
| | Compound 1 at a target concentration of 20 mg/mL |
| | (where the target concentration is expressed |
| | according to the amount of Compound 1 initially |
| | added to the formulation without regard to any |
| | conversion of Compound 1 to a salt form upon |
| | reaction with phosphate buffer or other basic |
| | species in the formulation) |
| | at pH 7.4, where sodium hydroxide was added to the |
| | formulation to achieve a pH of 7.4 for the formulation. |
| 7 | Aqueous formulation containing: |
| | Compound 1 at a target concentration of 10 mg/mL |
| | (where the target concentration is expressed |
| | according to the amount of Compound 1 initially |
| | added to the formulation without regard to any |
| | conversion of Compound 1 to a salt form upon |
| | reaction with phosphate buffer or other basic |
| | species in the formulation) |
| | at pH 7.4, where sodium hydroxide was added to |
| | the formulation to achieve a pH of 7.4 for |
| | the formulation. |

Each of formulations 1 to 7 above was evaluated by physical observation for the presence of particles in the formulation immediately after preparing the formulation. At a time that was immediately after preparing each of Formulations 1 to 7, each formulation was visually observed to be a clear solution without any visibly observable particles or fibers.

Multiple aliquots of each of Formulations 1 to 7 were stored in a vial for one week at a temperature of either 4° C., 25° C., or 40° C. After one week of storage, each vial was visually inspected for the presence of particles or fibers. Results are provided in Tables 2-4 below. The results show that a substantial number of vials contained particles or fibers after storage for one week at the stated temperature conditions (i.e., 4° C., 25° C., or 40° C.). Thus, Formulations 1 to 7 did not demonstrate satisfactory stability to storage.

TABLE 2
Results of Stability Study Conducted at 4° C.

| | | Number of Vials of Formulation |
|---|---|---|
| | Total Number of Vials of | Visually Observed to Contain |
| Formulation | Formulation Solution in | Particles or Fibers After |
| No. | Stability Study | Storage for One Week |
| 1 | 11 | 7 |
| 2 | 14 | 11 |
| 3 | 14 | 9 |
| 4 | 14 | 9 |
| 5 | 12 | 5 |
| 6 | 11 | 6 |
| 7 | 14 | 4 |

TABLE 3
Results of Stability Study Conducted at 25° C.

| | | Number of Vials of Formulation |
|---|---|---|
| | Total Number of Vials of | Visually Observed to Contain |
| Formulation | Formulation Solution in | Particles or Fibers After |
| No. | Stability Study | Storage for One Week |
| 1 | 13 | 9 |
| 2 | 13 | 12 |
| 3 | 14 | 11 |
| 4 | 14 | 6 |
| 5 | 12 | 2 |
| 6 | 10 | 4 |
| 7 | 14 | 6 |

TABLE 4
Results of Stability Study Conducted at 40° C.

| | | Number of Vials of Formulation |
|---|---|---|
| | Total Number of Vials of | Visually Observed to Contain |
| Formulation | Formulation Solution in | Particles or Fibers After |
| No. | Stability Study | Storage for One Week |
| 1 | 12 | 10 |
| 2 | 14 | 13 |
| 3 | 13 | 12 |
| 4 | 13 | 9 |
| 5 | 12 | 5 |
| 6 | 10 | 5 |
| 7 | 14 | 7 |

### Example 2—Stability Analysis of Aqueous Formulations Containing Compound 1 and a Polysorbate, Cremophor EL, or Cyclodextrin

Compound 1 was formulated in aqueous formulations, and the aqueous formulations were evaluated for stability. To evaluate stability of the formulations, the formulations were stored at 25° C. for a duration of one day or seven days, and then evaluated for visible appearance, concentration of Compound 1 in the formulation, and purity of the formulation according to liquid chromatographic analysis. The formulations contained Tween 20, Tween 80, Cremophor EL, or hydroxypropyl-beta-cyclodextrin (HPβCD). The concentration of Compound 1 in the formulation is expressed in units of mg/mL without regard to whether Compound 1 appears as the free acid or a salt form (e.g., a sodium salt of Compound 1).

Compound 1 has the following formula:

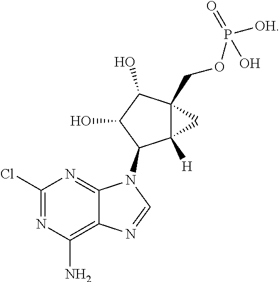 [SMILES: Nc1nc(Cl)nc2c1ncn2[C@H]1[C@H](O)[C@H](O)[C@]2(COP(=O)(O)O)C[C@H]12]

Results of the stability studies are set forth in Tables 5-8 below. The results show that many of the formulations were unstable to storage, due to the formation of a precipitate under the storage conditions used in the experiment.

TABLE 5
Stability Study Results from Storage at 25° C.

| | Initial Formulation | | | Formulation Characterization After | | |
|---|---|---|---|---|---|---|
| | Characterization (0 Days Storage) | | | Storage for 1 Day | | |
| | | Conc. of | Purity | | Conc. of | |
| Formulation | Formulation | Compound | (%) | Formulation | Compound | Purity |
| Excipients | Appearance | 1 (mg/mL) | | Appearance | 1 (mg/mL) | (%) |
| 10% Tween 80 | hazy | 2.20 | 99.63 | White suspension | 2.17 | 99.61 |
| in water | | | | with much | | |
| | | | | precipitant | | |
| 10% Tween 20 | hazy | 2.10 | 99.69 | White suspension | 2.09 | 99.59 |
| in water | | | | with much | | |
| | | | | precipitant | | |
| 10% | hazy | 2.37 | 99.67 | White suspension | 2.35 | 99.61 |
| Cremophor EL | | | | with much | | |
| in water | | | | precipitant | | |
| 10% HPβCD | clear | 2.65 | 99.71 | White | 2.52 | 99.58 |
| in water | | | | suspension | | |
| | | | | with a little | | |
| | | | | precipitant | | |

TABLE 6
Stability Study Results from Storage at 25° C.

| | Formulation Characterization | | | Formulation Characterization After | | |
|---|---|---|---|---|---|---|
| | After Storage for 4 Days | | | Storage for 7 Days | | |
| | | Conc. of | | | Conc. of | |
| Formulation | Formulation | Compound | | Formulation | Compound | |
| Excipients | Appearance | 1 (mg/mL) | Purity (%) | Appearance | 1 (mg/mL) | Purity (%) |
| 10% Tween 80 | White | 2.11 | 99.52 | clear | 2.08 | 99.51 |
| in water | suspension | | | | | |
| | with much | | | | | |
| | precipitant | | | | | |
| 10% Tween 20 | White | 1.98 | 99.51 | White suspension | 1.96 | 99.50 |
| in water | suspension | | | with plenty of | | |
| | with much | | | precipitant | | |
| | precipitant | | | | | |
| 10% Cremophor | White | 2.28 | 99.51 | White suspension | 2.33 | 99.53 |
| EL in water | suspension | | | with plenty of | | |
| | with much | | | precipitant | | |
| | precipitant | | | | | |
| 10% HPβCD | White | 2.58 | 99.55 | White suspension | 2.51 | 99.54 |
| in water | suspension | | | with plenty of | | |
| | with much | | | precipitant | | |
| | precipitant | | | | | |

TABLE 7
Stability Study Results from Storage at 40° C.

| | Formulation Characterization After | | | Formulation Characterization After | | |
|---|---|---|---|---|---|---|
| | Storage for 1 Day | | | Storage for 4 Days | | |
| | | Conc. of | | | Conc. of | |
| Formulation | Formulation | Compound 1 | | Formulation | Compound 1 | Purity |
| Excipients | Appearance | (mg/mL) | Purity (%) | Appearance | (mg/mL) | (%) |
| 10% Tween 80 | clear | 2.10 | 99.56 | clear | 2.02 | 99.42 |
| in water | | | | | | |
| 10% Tween 20 | White suspension | 2.10 | 99.52 | White suspension | 1.98 | 99.44 |
| in water | with much | | | with much | | |
| | precipitant | | | precipitant | | |
| 10% Cremophor | White suspension | 2.36 | 99.53 | White suspension | 2.28 | 99.44 |
| EL in water | with much | | | with much | | |
| | precipitant | | | precipitant | | |
| 10% HPβCD in | White suspension | 2.53 | 99.56 | White suspension | 2.53 | 99.47 |
| water | with much | | | with much | | |
| | precipitant | | | precipitant | | |

TABLE 8
Stability Study Results from Storage at 40° C.

Formulation Characterization After
Storage for 7 Days

| | | Conc. of | |
|---|---|---|---|
| Formulation | Formulation | Compound | Purity |
| Excipients | Appearance | 1 (mg/mL) | (%) |
| 10% Tween 80 in | clear | 2.07 | 99.00 |
| water | | | |
| 10% Tween 20 in | White suspension | 2.00 | 99.23 |
| water | with plenty of | | |
| | precipitant | | |
| 10% Cremophor | White suspension | 2.33 | 99.10 |
| EL in water | with plenty of | | |
| | precipitant | | |
| 10% HPβCD in | White suspension | 2.46 | 99.38 |
| water | with plenty of | | |
| | precipitant | | |

### Example 3—Stability Analysis of Aqueous Formulations Containing Compound 1 and a Polysorbate

Compound 1 was formulated in aqueous formulations containing Polysorbate 20, and the resulting aqueous formulations were evaluated for stability. To evaluate stability of the formulations, the formulations were stored for a duration of four weeks, and then the formulations were physically examined to identify any visible particles in the formulation. Samples of the formulations were stored at −20° C., 2° C. to 8° C., 25° C. at 60% relative humidity (RH), or 40° C. at 75% RH in this stability study. The concentration of Compound 1 in the formulation is expressed in units of mg/mL without regard to whether Compound 1 appears as the free acid or a salt form (e.g., a sodium salt of Compound 1).

Compound 1 has the following formula:

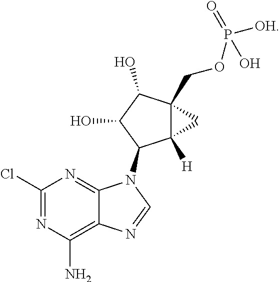 [SMILES: Nc1nc(Cl)nc2c1ncn2[C@H]1[C@H](O)[C@H](O)[C@]2(COP(=O)(O)O)C[C@H]12]

Results of the stability study are set forth in Tables 9 and 10 below. The results shows that particles formed in the formulation stored at 25° C. at 60% RH and in the formulation stored at 40° C. at 75% RH for at least two weeks. The formation of particles during storage indicates the formulations did not have good stability to storage.

TABLE 9
Stability Study Results

| | | | | Visible Particles | |
|---|---|---|---|---|---|
| | | Storage | | Initial | |
| | | Conditions for | | (0 Weeks | 1 Week of |
| No. | Formulation Description | Formulation | | Storage) | Storage |
| 1 | 2 mg/mL Compound 1, | −20° | C. | No particles | Not tested |
| | 0.02% (w/v) EDTA, | 2 to 8° | C. | No particles | No particles |
| | 0.5% (w/v) Polysorbate 20, | 25° C./60% RH | | No particles | No particles |
| | 20 mM citrate buffer, | 40° C./75% RH | | No particles | No particles |
| | 7% (w/v) sucrose, | | | | |
| | pH 8 | | | | |
| 2 | 5 mg/mL Compound 1, | −20° | C. | No particles | Not tested |
| | 0.02% (w/v) EDTA, | 2 to 8° | C. | No particles | No particles |
| | 0.5% (w/v) Polysorbate 20, | 25° C./60% RH | | No particles | No particles |
| | 20 mM citrate buffer, | 40° C./75% RH | | No particles | No particles |
| | 7 (w/v) sucrose | | | | |
| | pH 8 | | | | |

TABLE 10
Stability Study Results

| | | Storage | | Visible Particles | |
|---|---|---|---|---|---|
| | | Conditions for | | 2 Weeks of | 4 Weeks of |
| No. | Formulation | Formulation | | Storage | Storage |
| 1 | 2 mg/mL Compound 1, | −20° | C. | No particles | No particles |
| | 0.02% (w/v) EDTA, | 2 to 8° | C. | No particles | No particles |
| | 0.5% (w/v) Polysorbate 20, | 25° C./60% RH | | Have particles | Have particles |
| | 20 mM citrate buffer, | 40° C./75% RH | | Have particles | Have particles |
| | 7% (w/v) sucrose, | | | | |
| | pH 8 | | | | |
| 2 | 5 mg/mL Compound 1, | −20° | C. | No particles | No particles |
| | 0.02% (w/v) EDTA, | 2 to 8° | C. | No particles | No particles |
| | 0.5% (w/v) Polysorbate 20, | 25° C./60% RH | | Have particles | Have particles |
| | 20 mM citrate buffer, | 40° C./75% RH | | Have particles | Have particles |
| | 7 (w/v) sucrose | | | | |
| | pH 8 | | | | |

### Example 4—Stability Analysis of Additional Formulations Containing Compound 1 According to pH Value

Compound 1 was formulated in aqueous formulations containing EDTA, a citrate buffer, and sucrose, and the resulting aqueous formulations were evaluated for stability. To evaluate stability of the formulations, the formulations were stored for a duration of two weeks, and then evaluated for degradation, including physical examination to identify any visible particles in the formulation. Samples of the formulations were stored at 40° C. in the stability study. The concentration of Compound 1 in the formulation is expressed in units of mg/mL without regard to whether Compound 1 appears as the free acid or a salt form (e.g., a sodium salt of Compound 1). Each formulation contained 0.02% (w/v) EDTA, 20 mM of citrate buffer, 7% (w/v) sucrose, and the specific amount of Compound 1. Purity of the formulation was evaluated by liquid chromatography and expressed as percent purity.

Compound 1 has the following formula:

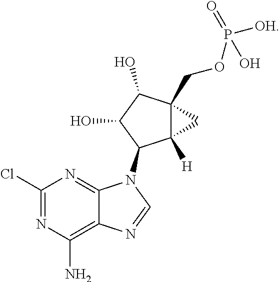 [SMILES: Nc1nc(Cl)nc2c1ncn2[C@H]1[C@H](O)[C@H](O)[C@]2(COP(=O)(O)O)C[C@H]12]

Results are provided in Tables 11 to 13 below.

TABLE 11
Formulation at Start of Stability Study (0 Weeks Storage)

| | | | Characterization of the Formulation | | | | | | | |
|---|---|---|---|---|---|---|---|---|---|---|
| | Target | | | | | | | | | Conc. |
| | Concentration | | | Sub Visible Particulates | | | | | | (mg/mL) |
| Sam- | of Compound 1 | For- | | >10 μm | >25 μm | | | Osmolality | | of |
| ple | in the | mulation | Visual | (no excess | (no excess | | | (mOsm/ | Purity | Com- |
| No. | Formulation | pH | inspection | 6000/vial) | 600/vial) | Result | pH | kg) | (%) | pound 1 |
| 1 | 0.5 mg/mL | pH 8.0 | No particles | 25 | 0 | pass | 7.98 | 273 | 99.63 | 0.51 |
| 2 | 2.5 mg/mL | | No particles | 2 | 0 | pass | 7.92 | 283 | 99.62 | 2.58 |
| 3 | 5 mg/mL | | No particles | 0 | 0 | pass | 7.94 | 301 | 99.63 | 5.19 |
| 4 | 10 mg/mL | | No particles | 0 | 0 | pass | 7.96 | 325 | 99.64 | 10.42 |
| 5 | 20 mg/mL | | No particles | 1 | 0 | pass | 7.9 | 363 | 99.62 | 20.14 |
| 6 | 5 mg/mL | pH 6.0 | No particles | 0 | 0 | pass | 5.9 | 296 | 99.59 | 5.31 |
| 7 | | pH 6.5 | No particles | 3 | 0 | pass | 6.45 | 297 | 99.60 | 5.19 |
| 8 | | pH 7.0 | No particles | 2 | 0 | pass | 7.02 | 307 | 99.62 | 5.34 |
| 9 | | pH 7.5 | No particles | 2 | 0 | pass | 7.52 | 304 | 99.62 | 5.39 |
| 10 | | pH 8.0 | No particles | 0 | 0 | pass | 7.98 | 305 | 99.61 | 5.48 |
| 11 | | pH 8.5 | No particles | 0 | 0 | pass | 8.45 | 300 | 99.62 | 5.39 |

TABLE 12
Formulation Stability Results at One Week of Storage

| | | | Characterization of the Formulation | | | | | | | |
|---|---|---|---|---|---|---|---|---|---|---|
| | Target | | | | | | | | | Conc. |
| | Concentration | | | Sub Visible Particulates | | | | | | (mg/mL) |
| Sam- | of Compound | For- | | >10 μm | >25 μm | | | | | of |
| ple | 1 in | mulation | Visual | (no excess | (no excess | | | Osmolality | Purity | Com- |
| No. | Formulation | pH | inspection | 6000/vial) | 600/vial) | Result | pH | (mOsm/kg) | (%) | pound 1 |
| 1 | 0.5 mg/mL | pH 8.0 | No particles | 2 | 0 | pass | 8.02 | 259 | 99.64 | 0.51 |
| 2 | 2.5 mg/mL | | No particles | 7 | 2 | pass | 8.01 | 273 | 99.65 | 2.57 |
| 3 | 5 mg/mL | | No particles | 0 | 0 | pass | 8.00 | 293 | 99.65 | 5.19 |
| 4 | 10 mg/mL | | No particles | 7 | 0 | pass | 8.00 | 314 | 99.64 | 10.29 |
| 5 | 20 mg/mL | | No particles | 12 | 0 | pass | 8.00 | 358 | 99.64 | 19.67 |
| 6 | 5 mg/mL | pH 6.0 | No particles | 5 | 0 | pass | 6.07 | 295 | 99.55 | 5.31 |
| 7 | | pH 6.5 | No particles | 2 | 0 | pass | 6.53 | 296 | 99.57 | 5.25 |
| 8 | | pH 7.0 | No particles | 7 | 2 | pass | 7.04 | 311 | 99.62 | 5.34 |
| 9 | | pH 7.5 | No particles | 2 | 0 | pass | 7.57 | 307 | 99.65 | 5.43 |
| 10 | | pH 8.0 | No particles | 0 | 0 | pass | 8.07 | 312 | 99.64 | 5.47 |
| 11 | | pH 8.5 | No particles | 2 | 0 | pass | 8.42 | 304 | 99.62 | 5.38 |

TABLE 13
Formulation Stability Results at Two Weeks of Storage

| | | | Characterization of the Formulation | | | | | | | |
|---|---|---|---|---|---|---|---|---|---|---|
| | | | | Sub Visible Particulates | | | | | | Conc. |
| | Target | | | | >25 μm | | | | | (mg/mL) |
| Sam- | Concentration | For- | | >10 μm | (no | | | | | of |
| ple | of Compound 1 | mulation | Visual | (no excess | excess | | | Osmolality | Purity | Compound |
| No. | in Formulation | pH | inspection | 6000/vial) | 600/vial) | Result | pH | (mOsm/kg) | (%) | 1 |
| 1 | 0.5 mg/mL | pH 8.0 | No particles | 2 | 0 | pass | 7.84 | 269 | 99.63 | 0.51 |
| 2 | 2.5 mg/mL | | No particles | 2 | 0 | pass | 8.04 | 283 | 99.63 | 2.58 |
| 3 | 5 mg/mL | | No particles | 7 | 0 | pass | 8.00 | 300 | 99.62 | 5.11 |
| 4 | 10 mg/mL | | No particles | 2 | 0 | pass | 8.03 | 327 | 99.63 | 10.43 |
| 5 | 20 mg/mL | | Have particles | 15 | 5 | pass | 8.03 | 366 | 99.62 | 21.63 |
| 6 | 5 mg/mL | pH 6.0 | No particles | 5 | 0 | pass | 6.01 | 295 | 99.39 | 5.31 |
| 7 | 5 mg/mL | pH 6.5 | No particles | 20 | 0 | pass | 6.49 | 297 | 99.45 | 5.23 |
| 8 | 5 mg/mL | pH 7.0 | No particles | 0 | 0 | pass | 7.02 | 308 | 99.55 | 5.38 |
| 9 | 5 mg/mL | pH 7.5 | No particles | 0 | 0 | pass | 7.58 | 30 | 99.61 | 5.45 |
| 10 | 5 mg/mL | pH 8.0 | No particles | 5 | 0 | pass | 8.11 | 300 | 99.63 | 5.49 |
| 11 | 5 mg/mL | pH 8.5 | No particles | 0 | 0 | pass | 8.41 | 300 | 99.64 | 5.38 |

### Example 5—Preparation of Additional Formulation Containing Compound 1

Compound 1 was formulated in an aqueous formulation containing EDTA sodium salt, a citrate buffer, and sucrose, and the resulting aqueous formulation was evaluated for stability. Stability of the formulation was evaluated in a four-week stability study. Results are provided below. Compound 1 has the following formula:

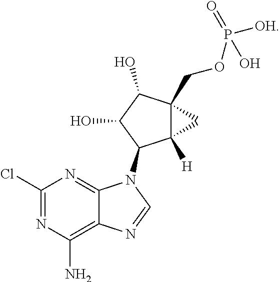 [SMILES: Nc1nc(Cl)nc2c1ncn2[C@H]1[C@H](O)[C@H](O)[C@]2(COP(=O)(O)O)C[C@H]12]

### Part I—Preparation of Aqueous Formulation

The formulation set forth in Table 14 below was prepared according to the following procedure, where the concentration of Compound 1 in the formulation is expressed in units of mg/mL without regard to whether Compound 1 appears as the free acid or a salt form (e.g., a sodium salt of Compound 1).

To achieve a 100 mL amount of the formulation: 80 mL of water was added to a bottle with a rotor. 514.06 mg of sodium citrate dihydrate was added to the water in the bottle. To the resulting mixture was added 53 mg of citric acid monohydrate. To the resulting mixture was added a 2.7 mL aliquot of 1M NaOH.

Next, 20 mg of EDTA was added to the mixture, followed by 6.9 g of sucrose, and the resulting solution was mixed well. To the resulting solution was then added 50 mg of Compound 1. As needed, the pH of the resulting solution was adjusted to a pH of 8.0±0.2 by adding to the solution an aliquot(s) of 0.1M NaOH or 0.1M HCl. Purified water was added to the solution in order to achieve 100 mL total volume. After confirming the final pH, the resulting solution was subjected to filtration through a 0.22 μm filter, thereby providing the final aqueous formulation.

TABLE 14
Aqueous Formulation

| | | |
|---|---|---|
| Component | Concentration | Concentration |

| | | | |
|---|---|---|---|
| Compound 1 | 5.00 | mg/mL | 0.50% (w/v) |
| Buffer containing tri-sodium | 20 | mM | 20 mM |
| citrate and citric acid | | | |
| Sucrose | 69.00 | mg/mL | 6.90% (w/v) |
| Disodium Edetate | 0.20 | mg/mL | 0.02% (w/v) |
| Sodium hydroxide | 1.08 | mg/mL | 0.11% (w/v) |

| | |
|---|---|
| Sodium hydroxide - Added as | As needed |

0.1N Solution to Adjust pH

| | |
|---|---|
| Hydrochloric acid - Added as | As needed |

0.1N Solution to Adjust pH

| | |
|---|---|
| Target pH | 8.0 ± 0.5 |

| | | |
|---|---|---|
| Water | q.s | q.s |

### Part II—Analysis of Formulation Stability to Storage

The aqueous formulation set forth in Table 14 above was analyzed for stability to storage over a duration of four weeks. Samples of the aqueous formulation were stored at one of the following temperatures: −20° C., a temperature in the range of 2° C. to 8° C., 25° C., or 40° C.

Results of the stability study are provided in the tables below. The formulation demonstrated good stability to storage under the conditions tested.

TABLE 15
Stability Study Results

| | | | | | Formulation Purity | |
|---|---|---|---|---|---|---|
| | | | | | by Liquid | Measured |
| | | Duration of | | Osmolality of | Chromatography | Concentration |
| Storage | | Storage | pH of | Formulation | Analysis | of Compound 1 |
| Temperature | | (weeks) | Formulation | (mOsm/kg) | (%) | (mg/mL) |
| −20° | C. | 1 | 7.89 | 310 | 99.64 | 5.12 |
| 2 to 8° | C. | 1 | 7.90 | 315 | 99.61 | 5.11 |
| 25° | C. | 1 | 7.93 | 318 | 99.62 | 5.14 |
| 40° | C. | 1 | 8.00 | 320 | 99.61 | 5.13 |
| −20° | C. | 2 | 7.95 | 311 | 99.65 | 5.12 |
| 2 to 8° | C. | 2 | 7.95 | 316 | 99.63 | 5.12 |
| 25° | C. | 2 | 7.99 | 317 | 99.65 | 5.11 |
| 40° | C. | 2 | 8.05 | 315 | 99.63 | 5.12 |
| −20° | C. | 4 | 7.91 | 319 | 99.62 | 5.10 |
| 2 to 8° | C. | 4 | 7.91 | 316 | 99.64 | 5.09 |
| 25° | C. | 4 | 7.95 | 320 | 99.64 | 5.12 |
| 40° | C. | 4 | 8.05 | 320 | 99.65 | 5.12 |

TABLE 16
Stability Study Results

| | | | | Sub Visible Particulates | | |
|---|---|---|---|---|---|---|
| | | Duration | Visual | >10 μm | >25 μm | |
| Storage | | of Storage | Inspection of | (no excess | (no excess | |
| Temperature | | (weeks) | Formulation | 6000/vial) | 600/vial) | Result |
| −20° | C. | 1 | No particles | 2 | 0 | pass |
| 2 to 8° | C. | 1 | No particles | 2 | 2 | pass |
| 25° | C. | 1 | No particles | 0 | 0 | pass |
| 40° | C. | 1 | No particles | 7 | 5 | pass |
| −20° | C. | 2 | No particles | 5 | 0 | pass |
| 2 to 8° | C. | 2 | No particles | 7 | 0 | pass |
| 25° | C. | 2 | No particles | 5 | 0 | pass |
| 40° | C. | 2 | No particles | 2 | 0 | pass |
| −20° | C. | 4 | No particles | 2 | 0 | pass |
| 2 to 8° | C. | 4 | No particles | 0 | 0 | pass |
| 25° | C. | 4 | No particles | 5 | 0 | pass |
| 40° | C. | 4 | No particles | 2 | 0 | pass |

### Example 6—Stability Analysis of an Exemplary Formulation When Combined With A Diluent for Intravenous Administration

The formulation in Table 17 below was diluted with either saline solution or Lactated Ringer's solution to provide a test solution containing Compound 1 at a concentration of 0.4 mg/mL. The concentration of Compound 1 in the formulation is expressed in units of mg/mL without regard to whether Compound 1 appears as the free acid or a salt form (e.g., a sodium salt of Compound 1). The test solution was stored for 128 hours, and then evaluated for stability. Compound 1 has the following formula:

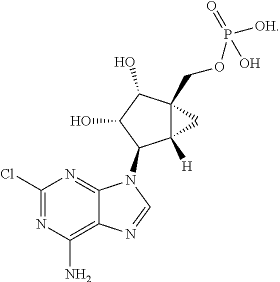 [SMILES: Nc1nc(Cl)nc2c1ncn2[C@H]1[C@H](O)[C@H](O)[C@]2(COP(=O)(O)O)C[C@H]12]

TABLE 17
Aqueous Formulation

| | | |
|---|---|---|
| Component | Concentration | Concentration |

| | | | |
|---|---|---|---|
| Compound 1 | 5.00 | mg/mL | 0.50% (w/v) |
| Buffer containing tri-sodium | 20 | mM | 20 mM |
| citrate and citric acid | | | |
| Sucrose | 69.00 | mg/mL | 6.90% (w/v) |
| Disodium Edetate | 0.20 | mg/mL | 0.02% (w/v) |
| Sodium hydroxide | 1.08 | mg/mL | 0.11% (w/v) |

| | |
|---|---|
| Sodium hydroxide - Added as | As needed |

0.1N Solution to Adjust pH

| | |
|---|---|
| Hydrochloric acid - Added as | As needed |

0.1N Solution to Adjust pH

| | |
|---|---|
| Target pH | 8.0 ± 0.5 |

| | | |
|---|---|---|
| Water | q.s | q.s |

The results show that both test solutions had good stability, and that the formulation in Table 17 above is compatible with saline solution and Lactated Ringer's solution at the conditions tested.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. An aqueous formulation, comprising:
(a) from 0.25% (w/v) to 0.75% (w/v) of a compound of Formula I, wherein Formula I is represented by: 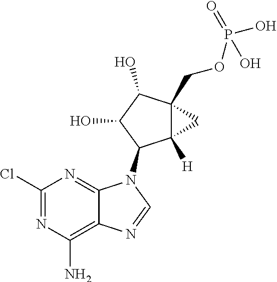 [SMILES: Nc1nc(Cl)nc2c1ncn2[C@H]1[C@H](O)[C@H](O)[C@]2(COP(=O)(O)O)C[C@H]12]
or a pharmaceutically acceptable salt thereof;
(b) from 0.01% (w/v) to 0.03% (w/v) of a chelating agent selected from ethylenediamine tetraacetic acid, a pharmaceutically acceptable salt of ethylenediamine tetraacetic acid, or a mixture thereof;
(c) from 10 mM to 30 mM of a buffer comprising citric acid and a pharmaceutically acceptable salt of citric acid;
(d) from 3.5% (w/v) to 11% (w/v) of a tonicity modifier; and
(e) at least 90% (w/v) water; and
having a pH in the range of 7.5 to 8.5.

2. The formulation of claim 1, wherein the compound of Formula I is present in an amount of from 0.4% (w/v) to 0.6% (w/v).

3. The formulation of claim 1, wherein the compound of Formula I is present in an amount of about 0.5% (w/v).

4. (canceled)

5. (canceled)

6. The formulation of claim 1, wherein the compound of Formula I is an alkali metal or alkaline earth metal pharmaceutically acceptable salt of

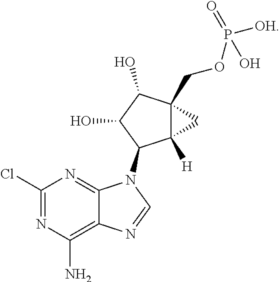 [SMILES: Nc1nc(Cl)nc2c1ncn2[C@H]1[C@H](O)[C@H](O)[C@]2(COP(=O)(O)O)C[C@H]12]

7. The formulation of claim 1, wherein the compound of Formula I is a sodium salt of

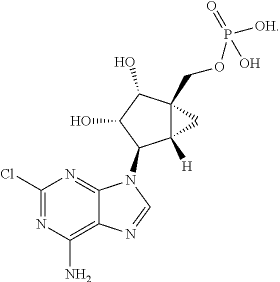 [SMILES: Nc1nc(Cl)nc2c1ncn2[C@H]1[C@H](O)[C@H](O)[C@]2(COP(=O)(O)O)C[C@H]12]

8. (canceled)

9. The formulation of claim 1, wherein the formulation comprises about 0.02% (w/v) of a chelating agent selected from ethylenediamine tetraacetic acid, a pharmaceutically acceptable salt of ethylenediamine tetraacetic acid, or a mixture thereof.

10. (canceled)

11. (canceled)

12. The formulation of claim 1, wherein the formulation comprises about 20 mM of a buffer comprising citric acid and a pharmaceutically acceptable salt of citric acid.

13. (canceled)

14. (canceled)

15. The formulation of claim 1, wherein the formulation comprises about 6.9% (w/v) of a tonicity modifier.

16. (canceled)

17. The formulation of claim 1, wherein the tonicity modifier is a disaccharide.

18. (canceled)

19. The formulation of claim 1, wherein the formulation has a pH in the range of 7.8 to 8.2.

20. (canceled)

21. (canceled)

22. (canceled)

23. (canceled)

24. The formulation of claim 1, wherein the formulation comprises at least 91% water.

25. An aqueous formulation, comprising:
(a) about 0.5% (w/v) of a compound of Formula I, wherein Formula I is represented by: 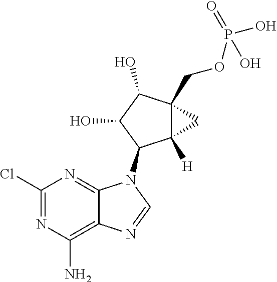 [SMILES: Nc1nc(Cl)nc2c1ncn2[C@H]1[C@H](O)[C@H](O)[C@]2(COP(=O)(O)O)C[C@H]12]
or a pharmaceutically acceptable salt thereof;
(b) about 0.02% (w/v) of a chelating agent selected from ethylenediamine tetraacetic acid, a pharmaceutically acceptable salt of ethylenediamine tetraacetic acid, or a mixture thereof,
(c) about 20 mM of a buffer comprising citric acid and a pharmaceutically acceptable salt of citric acid;
(d) about 6.9% (w/v) of a tonicity modifier; and
(e) at least 90% (w/v) water; and
having a pH of about 8.

26. An aqueous formulation, comprising:
(a) about 0.5% (w/v) of a compound of Formula I, wherein Formula I is represented by: 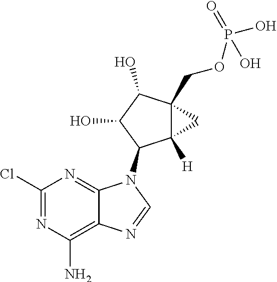 [SMILES: Nc1nc(Cl)nc2c1ncn2[C@H]1[C@H](O)[C@H](O)[C@]2(COP(=O)(O)O)C[C@H]12]
or a pharmaceutically acceptable salt thereof;
(b) about 0.02% (w/v) of a chelating agent selected from ethylenediamine tetraacetic acid, a pharmaceutically acceptable salt of ethylenediamine tetraacetic acid, or a mixture thereof,
(c) about 20 mM of a buffer comprising citric acid and a pharmaceutically acceptable salt of citric acid;
(d) about 6.9% (w/v) sucrose; and
(e) at least 90% (w/v) water; and
having a pH of about 8.

27. The formulation of claim 26, wherein the formulation comprises:
(a) 0.5% (w/v) of a compound of Formula I, wherein Formula I is represented by: 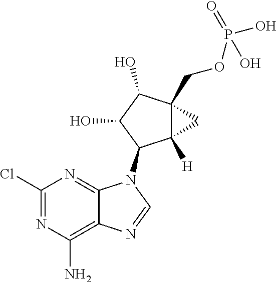 [SMILES: Nc1nc(Cl)nc2c1ncn2[C@H]1[C@H](O)[C@H](O)[C@]2(COP(=O)(O)O)C[C@H]12]
or a pharmaceutically acceptable salt thereof;
(b) 0.02% (w/v) of a chelating agent selected from ethylenediamine tetraacetic acid, a pharmaceutically acceptable salt of ethylenediamine tetraacetic acid, or a mixture thereof;
(c) 20 mM of a buffer comprising citric acid and a pharmaceutically acceptable salt of citric acid;
(d) 6.9% (w/v) sucrose; and
(e) at least 90% (w/v) water; and
having a pH of 8.

28-57. (canceled)

58. The formulation of claim 25, wherein the formulation has an osmolality in the range from about 270 mOsm/kg to about 330 mOsm/kg.

59. (canceled)

60. The formulation of claim 25, wherein less than 1% by weight of compound of Formula I degrades upon storage of the formulation at 25° C. for 4 weeks.

61. (canceled)

62. (canceled)

63. (canceled)

64. (canceled)

65. The formulation of claim 25, wherein less than 0.5% by weight of compound of Formula I degrades upon storage of the formulation at 40° C. for 4 weeks.

66. The formulation of claim 25, wherein the formulation contains less than 0.02% (w/v) of the following compound or a pharmaceutically acceptable salt thereof:

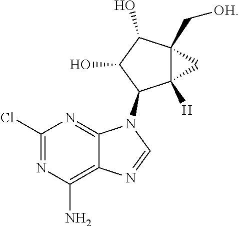 [SMILES: Nc1nc(Cl)nc2c1ncn2[C@H]1[C@H](O)[C@H](O)[C@]2(CO)C[C@H]12]

67. (canceled)

68. (canceled)

69. (canceled)

70. (canceled)

71. (canceled)

72. An aqueous injectable formulation, comprising a formulation of claim 1 and a diluent for intravenous administration.

73. An aqueous injectable formulation produced by admixing a formulation of claim 1 and a diluent for intravenous administration.

74. A method of preparing an aqueous injectable formulation, comprising admixing a formulation of claim 1 and a diluent for intravenous administration.

75. A method for treating a cardiac disorder, comprising administering to a subject in need thereof a therapeutically effective amount of an aqueous injectable formulation of claim 73 to treat the cardiac disorder.

76. A method for improving cardiac contractile performance in a subject, comprising administering to a subject in need thereof an effective amount of an aqueous injectable formulation of claim 73 to improve cardiac contractile performance.

77. A method for improving cardiac function in a subject, comprising administering to a subject in need thereof an effective amount of an aqueous injectable formulation of claim 73 to improve cardiac function.

78-84. (canceled)
